(19) United States
(12) Patent Application Publication
(10) Pub. No.: US 2018/0070875 A1
(43) Pub. Date: Mar. 15, 2018

(54) Determining Stem Cell Treatment Effectiveness By Monitoring Physical Progress

(71) Applicant: Simren Kshetrapal, Berkeley, CA (US)

(72) Inventor: Simren Kshetrapal, Berkeley, CA (US)

(21) Appl. No.: 15/266,817

(22) Filed: Sep. 15, 2016

(51) Int. Cl. A61B 5/00; A61K 35/545; A61B 5/11; A61B 5/03; A61B 5/04; A61B 5/0205; A41D 13/12; A61B 5/026; G06F 19/00

(52) U.S. Cl. CPC A61B 5/4848; A61B 2505/09; A61B 5/1125; A61B 5/031; A61B 5/04001; A61B 5/0205; A41D 13/1263; A61B 5/6804; A61B 5/0261; A61B 5/743; A61B 5/748; A61B 5/1118; A61B 5/4839; A61B 5/0013; G06F 19/3481; G06F 19/325; G06F 19/3406; A61B 2562/0219; A61B 2562/028; A61B 5/0073; A61K 35/545

(57) ABSTRACT

A method for monitoring physical progress of a target object undergoing stem cell treatment (SCT) for determining SCT effectiveness and adapting the SCT is provided. Motion sensors within a wearable motion monitoring device configured as a body suit monitor micro-movements of body parts of the target object on a time basis. Micro-movement data is recorded, tabulated, and summarized on the time basis. The method employs an SCT monitoring system (SCTMS) that records physiological condition parameters of the target object in a predefined stem cell treatment checklist using user inputs, neural activity data received from neural sensors, and motor activity data received from the motion sensors. The SCTMS performs a baseline analysis of the physical progress of the target object based on the physiological condition parameters. The SCTMS generates and renders a physical progress report and a stem cell treatment plan based on the baseline analysis on an electronic device.

### BACKGROUND

Regenerative medicine deals with a process of replacing, engineering, and regenerating damaged, diseased and defective cells, tissues, and organs of a human body by stimulating the human body's own repair mechanism. Some biomedical approaches of regenerative medicine involve the use of stem cells to treat or prevent a medical condition involving, for example, spinal cord injuries, neurological disease, Lyme disease, etc., of a patient. Stem cells are undifferentiated cells that are capable of giving rise to more cells of the same type, and from which other kinds of cells arise by differentiation. Stem cells are found within tissues and organs throughout the human body. The stem cells within niche tissues and organs serve the purpose of regeneration. For example, when cells present in tissues of the stomach are damaged or diseased, stem cells within niche tissues of the stomach undergo mitotic division to reproduce cells necessary to replace the damaged or diseased cells in the stomach. A typical stem cell treatment involves a physician injecting stem cells into the blood supply and/or proximal to a location of injury of a patient. The injected stem cells work by providing new cells to replace damaged, diseased and defective cells to treat or prevent a medical condition of the patient. As the injected stem cells mature, the stem cells replace and assimilate to the function of the damaged, diseased and defective cells. The physician may prescribe additional physical therapy and/or behavioral therapy to monitor gross motor skills, fine motor skills, etc., as part of the patient's stem cell treatment to measure and/or determine the effectiveness of the stem cell treatment.

In conventional stem cell treatment methods, a physician administers the stem cells to an affected body part of the patient and prescribes physical therapy and/or behavioral therapy as part of the stem cell treatment. Physical therapy and/or behavioral therapy administered to the patient trains the stem cells to mature into the correct body cells. For example, when a patient undergoing stem cell treatment for a spinal cord injury performs back exercises, the stem cells administered proximal to the spinal cord mature into cells localized to the spinal cord. The physician manually and visually monitors, measures, and records physical progress of the patient subjected to the physical therapy and/or the behavioral therapy to determine a baseline and effectiveness of the stem cell treatment administered to the patient. Conventional methods of monitoring, measuring, and recording the physical progress of the patient are subjective as they involve visual assessment of the progress of the patient by the physician, for example, by viewing how much further the patient can move his/her leg, or neck, or spine, etc., and are not based on real time data, for example, real time physical progress data of the patient, resulting in generation of incomplete and/or inoperative data. The incomplete and/or inoperative data leads to a less than optimal treatment of the medical condition of the patient undergoing the stem cell treatment. Moreover, conventional stem cell treatment methods do not provide information required to determine the effectiveness of the stem cell treatment administered to the patient.

In conventional stem cell treatment methods, the physician manually diagnoses the medical condition of the patient undergoing the stem cell treatment based on his/her prognosis and/or experience. The conventional stem cell treatment methods employed by the physician to diagnose the medical condition of the patient are often based on tradition and/or authority. Hence, in conventional stem cell treatment methods, the diagnosis of the patient is dependent on the knowledge of the physician to assess the medical condition of the patient resulting in inaccurate and/or ineffective diagnosis and treatment of the medical condition of the patient undergoing the stem cell treatment. The conventional stem cell treatment methods are not standardized and do not provide an objective and concise set of guidelines on how to diagnose and measure the physical progress and/or the condition of the patient.

Conventional procedures for measuring motion of a patient are rudimentary. For example, in conventional procedures, the physician measures the progress of the patient, for example, with a measuring tape, a ruler or a similar motion device. This type of measurement is subjective for both macro and micro-movements such as handwriting movements and is not accurate. Reporting in these conventional procedures is manual and based on accuracy of notes of a preparer. The measured data is also not tabulated or graphed and the dosage of stem cells is generally administered subjectively.

In some conventional stem cell treatment methods, sensor systems are implemented to monitor, measure, and record the physical progress of the patient subjected to physical therapy. These sensor systems are typically connected to different individual body parts of the body of the patient to monitor, measure, and record motor activity data and neural activity data of the patient undergoing the stem cell treatment. Electrodes of external sensor systems used in conventional stem cell treatment methods are typically connected to individual body parts of the patient to measure and record the physical progress of the patient. These external sensor systems do not conform to the body of the patient and therefore generate contact based errors. These external sensor systems also cannot accurately measure motor activity data comprising, for example, micro-movements such as fine motor skills, gross motor skills, etc., of the patient undergoing the stem cell treatment. Conventional neural sensors used in the conventional stem cell treatment methods are not capable of accurately measuring and detecting the neural activity data, for example, vascular damage caused by a brain injury, vascular damage caused by a spinal cord injury, cerebral fluid flow, blood flow in the brain, etc., of the patient undergoing the stem cell treatment.

The conventional stem cell treatment comprises a manual, subjective analysis of a patient for monitoring, measuring, and recording the physical progress of the patient to determine the effectiveness of the stem cell treatment administered to the patient, which is susceptible to human errors. Furthermore, a physician typically adapts the stem cell treatment based on a visual assessment or a manual assessment of the patient. The visual assessment or the manual assessment used in the conventional stem cell treatment methods does not accurately measure subtle changes in a disease or an injury of the patient to determine the effectiveness of the stem cell treatment administered to the patient. The physician with incomplete data or inaccurate data cannot adapt the stem cell treatment to provide an effective stem cell treatment to the disease or the injury of the patient.

Hence, there is a long felt need for a method for optimally monitoring and measuring micro-movements of a target object, for example, a patient undergoing stem cell treatment, for determining effectiveness of the stem cell treatment and adapting the stem cell treatment administered to the target object. Moreover, there is a need for a method and a system for monitoring physical progress of a target object undergoing stem cell treatment by accurately measuring and analyzing motor activity data and neural activity data of the target object using precise sensors and wearable motion monitoring devices configured, for example, as full body suits that measure a range of motion in real time, in combination with an electronic device, for example, a tablet computing device, a laptop, etc. Furthermore, there is a need for a method and a system that provide condition based checklists that define a set of guidelines for diagnosing medical conditions of the target object.

### SUMMARY OF THE INVENTION

This summary is provided to introduce a selection of concepts in a simplified form that are further disclosed in the detailed description of the invention. This summary is not intended to determine the scope of the claimed subject matter.

The method disclosed herein address the above recited need for optimally monitoring and measuring micro-movements of a target object, for example, a patient undergoing stem cell treatment, for determining effectiveness of the stem cell treatment and adapting the stem cell treatment administered to the target object. In one implementation, the method and the system disclosed herein address the above recited need for monitoring physical progress of a target object undergoing the stem cell treatment by accurately measuring and analyzing motor activity data and neural activity data of the target object using precise sensors and wearable motion monitoring devices configured, for example, as full body suits that measure range of motion in real time, in combination with an electronic device, for example, a tablet computing device, a laptop, etc. The method and the system disclosed herein also provide condition based stem cell treatment checklists that define a set of guidelines for diagnosing medical conditions of the target object. The method and the system disclosed herein monitor the physical progress of the target object using a feedback control loop formed between the sensors and the electronic device. The method and the system disclosed herein implement a combination of next generation wearable motion monitoring devices for recording motion in a more granular manner and the condition based stem cell treatment checklists to ensure that a medical practitioner, for example, a physician, a doctor, a caregiver, etc., does not miss a diagnosis and ongoing monitoring, that is, capturing a physical progress improvement of the target object, while the target object undergoes the stem cell treatment.

In the method for monitoring physical progress of a target object undergoing stem cell treatment for determining effectiveness of the stem cell treatment and adapting the stem cell treatment disclosed herein, micro-movement data of one or more body parts of the target object, micro-movements of which need to be monitored on a time basis, is recorded by three-dimensional (3D) motion sensors positioned within a wearable motion monitoring device configured as a body suit. The 3D motion sensors are positioned over the body parts to be monitored. The 3D motion sensors monitor the micro-movements of the body parts of the target object. The micro-movement data comprises data on the micro-movements comprising, for example, fine motor skills, gross motor skills, a fluid flow in a brain of the target object, an ultra-micro measurement for nerve activity of a spine of the target object, a range of motion restored, etc., of the body parts of the target object. The micro-movement data is recorded corresponding to a predefined condition based stem cell treatment checklist. The recorded micro-movement data collected by the 3D motion sensors is tabulated and summarized on the time basis, for example, by day, week, and month. The tabulated and summarized micro-movement data allows the medical practitioner to adapt the stem cell treatment by adapting a dosage and locations of administration of stem cells in the target object for improved care.

In one implementation, the method disclosed herein employs a stem cell treatment monitoring system (SCTMS) executable by at least one processor configured to execute computer program instructions for monitoring physical progress of a target object undergoing stem cell treatment for determining effectiveness of the stem cell treatment and adapting the stem cell treatment. The SCTMS renders a predefined condition based stem cell treatment checklist on a graphical user interface provided by the stem cell treatment monitoring system deployed on an electronic device. The SCTMS dynamically receives neural activity data and motor activity data of the target object from multiple sensors in operable communication with the SCTMS on the electronic device. The sensors comprise multiple neural sensors and a wearable motion monitoring device. The neural sensors are positioned proximal to a neural system of the target object. The neural sensors measure the neural activity data comprising a change of a fluid flow and an ultra-micro measurement of nerve activity in the neural system of the target object. The wearable motion monitoring device is configured as a body suit to conform to a body of the target object. The wearable motion monitoring device comprises motion sensors for measuring the motor activity data comprising data on micro-movements of one or more different body parts of the body of the target object.

The stem cell treatment monitoring system (SCTMS) records physiological condition parameters of the target object in the rendered predefined condition based stem cell treatment checklist using user inputs and the dynamically received neural activity data and the dynamically received motor activity data. The SCTMS performs a baseline analysis of the physical progress of the target object undergoing the stem cell treatment based on the recorded physiological condition parameters in real time. The SCTMS generates a physical progress report based on the baseline analysis of the physical progress of the target object for determining the effectiveness of the stem cell treatment. The SCTMS renders the generated physical progress report on the graphical user interface (GUI) displayed on the electronic device. The SCTMS also generates and renders a stem cell treatment plan based on the generated physical progress report of the target object on the GUI displayed on the electronic device.

In one or more embodiments, related systems comprise circuitry and/or programming for effecting the methods disclosed herein; the circuitry and/or programming can be any combination of hardware, software, and/or firmware configured to effect the methods disclosed herein depending upon the design choices of a system designer. Also, various structural elements can be employed depending on the design choices of the system designer.

### BRIEF DESCRIPTION OF THE DRAWINGS

The foregoing summary, as well as the following detailed description of the invention, is better understood when read in conjunction with the appended drawings. For the purpose of illustrating the invention, exemplary constructions of the invention are shown in the drawings. However, the invention is not limited to the specific methods and components disclosed herein. The description of a method step or a component referenced by a numeral in a drawing is applicable to the description of that method step or component shown by that same numeral in any subsequent drawing herein.

FIG. 1 illustrates a method for monitoring physical progress of a target object undergoing stem cell treatment for determining effectiveness of the stem cell treatment and adapting the stem cell treatment.

FIG. 2 illustrates an embodiment of the method for monitoring physical progress of a target object undergoing stem cell treatment for determining effectiveness of the stem cell treatment and adapting the stem cell treatment.

FIG. 3 exemplarily illustrates a schematic representation of the embodiment of the method for monitoring physical progress of a target object undergoing stem cell treatment for determining effectiveness of the stem cell treatment and adapting the stem cell treatment.

FIGS. 4A-4B exemplarily illustrate predefined condition based stem cell treatment checklists used for recording physiological condition parameters of a target object undergoing stem cell treatment.

FIG. 5 exemplarily illustrates a wearable motion monitoring device configured as a full body suit comprising multiple motion sensors positioned on locations corresponding to different body parts of a body of a target object.

FIGS. 6A-6B exemplarily illustrate tables indicating positioning of a combination of motion sensors on locations corresponding to different body parts of a body of a target object.

FIGS. 7A-7B exemplarily illustrate tables containing descriptions of movements related to activities of daily living of a target object undergoing stem cell treatment, to be monitored.

FIGS. 8A-8B exemplarily illustrate tables containing accuracy performances of multiple machine learning algorithms based on a combination of motion sensors.

FIGS. 9A-9B exemplarily illustrate confusion matrix tables containing optimal results of double, triple, quadruple and quintuple motion sensor configurations under individual machine learning algorithms.

FIG. 10 exemplarily illustrates a table containing k-nearest neighbor classifier results of ten successive measurements made by a motion sensor positioned on a waist of a target object.

FIGS. 11A-11B exemplarily illustrate tables containing a summary of the motion sensors, specifications of the motion sensors, combinations of the motion sensors, machine learning algorithms executed for measuring motor activity data of a target object undergoing stem cell treatment, and performances of the machine learning algorithms.

FIGS. 12A-12B exemplarily illustrate single-photon emission computed tomographic images of a brain of a target object undergoing stem cell treatment used by a stem cell treatment monitoring system for monitoring physical progress of the target object.

FIGS. 13A-13C exemplarily illustrate supplementary data of a target object used by the stem cell treatment monitoring system for monitoring physical progress of the target object.

FIG. 14 exemplarily illustrates initial assessment data generated by the stem cell treatment monitoring system during performance of a baseline analysis of physical progress of a target object undergoing stem cell treatment.

FIG. 15 exemplarily illustrates a physical progress report generated by the stem cell treatment monitoring system based on the baseline analysis of physical progress of a target object undergoing stem cell treatment.

FIG. 16 exemplarily illustrates a stem cell treatment plan generated by the stem cell treatment monitoring system based on a physical progress report of a target object undergoing stem cell treatment.

FIG. 17 exemplarily illustrates a system comprising sensors and the stem cell treatment monitoring system implemented on an electronic device for monitoring physical progress of a target object undergoing stem cell treatment for determining effectiveness of the stem cell treatment and adapting the stem cell treatment.

### DETAILED DESCRIPTION OF THE INVENTION

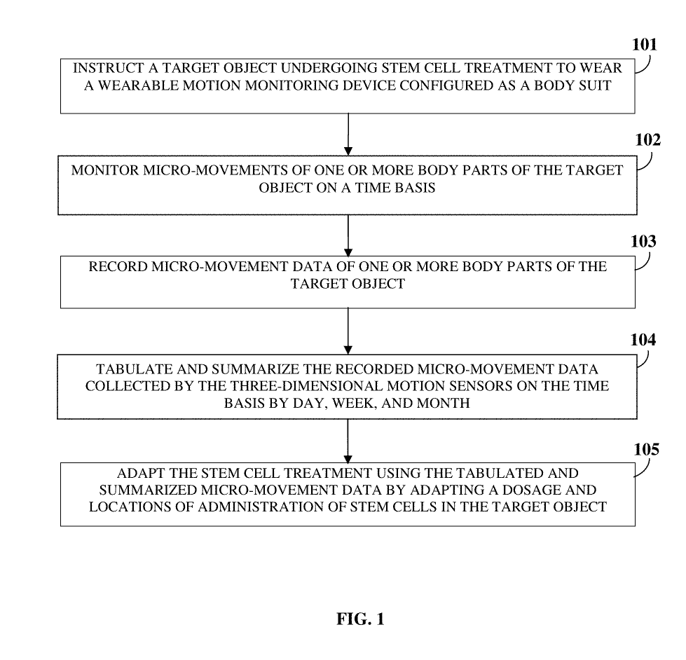

FIG. 1 illustrates a method for monitoring physical progress of a target object undergoing stem cell treatment for determining effectiveness of the stem cell treatment and adapting the stem cell treatment. As used herein, “physical progress” refers to an improvement in a medical condition of a target object undergoing stem cell treatment over a duration of time, for example, an ability of a patient to move a certain part of the patient's body which was not movable prior to the stem cell treatment. Also, as used herein, “medical condition” refers to a disease, an injury, a physiological condition, a disorder, etc. The medical condition is, for example, a spinal cord injury, a brain injury, a neurological disease, Lyme disease, a developmental disorder such as Asperger syndrome, Autism, etc. Also, as used herein, “target object” refers to an individual such as a person, an animal, etc., having a medical condition and undergoing stem cell treatment. In the stem cell treatment, stem cells are used to treat or prevent a medical condition.

In the method disclosed herein, a medical practitioner, for example, a doctor instructs 101 a target object, for example, a patient undergoing stem cell treatment to wear a wearable motion monitoring device configured as a body suit that conforms to the body of the target object. The wearable motion monitoring device is, for example, the Xsens MVN Biomech full body measurement body suit of Xsens Technologies B.V. Corporation, Enschede, Netherlands. Three-dimensional (3D) motion sensors positioned within the wearable motion monitoring device are positioned over one or more body parts of the target object to be monitored. Micro-movements of one or more body parts of the target object are monitored 102 using the 3D motion sensors on a time basis, for example, every 10 minutes. The micro-movements of the body parts of the target object comprise fine motor skills, gross motor skills, a fluid flow in the brain of the target object, ultra-micro measurement for nerve activity of a spine of the target object, and a range of motion restored. The fine motor skills refer to coordination of small muscles in movements involving synchronization of, for example, hands and fingers with eyes of the target object. The fine motor skills of the target object comprise, for example, hand-eye coordination, a range of motion of fingers of the target object, picking up objects between a thumb and a finger of the target object, writing, etc. The gross motor skills refer to movement and coordination of large body parts of the target object, for example, arms, hands, wrists, legs, feet, toes, etc., of the target object. In the method disclosed herein, micro-movement data of one or more body parts of the target object undergoing the stem cell treatment is recorded 103 by the 3D motion sensors. The micro-movement data comprises data on micro-movements of the body parts of the target object, that is, data on movements and actions of muscles of the body parts of the target object. In an embodiment, the 3D motion sensors monitor and record neural micro-movements in the range of, for example, about 1 micron to about 100 microns, muscle micro-movements in the range of, for example, about 1 micron to about 10,000 microns, and spinal and limb micro-movements in the range of, for example, about 0.1 centimeter (cm) to about 10 centimeters.

In an embodiment, the micro-movement data is recorded corresponding to a predefined condition base stem cell treatment checklist. As used herein, “predefined condition based stem cell treatment checklist” refers to an assessment tool that comprises a set of guidelines to be checked or examined for evaluating and/or diagnosing a predefined medical condition of a target object. For example, the predefined condition based stem cell treatment checklist for diagnosing Asperger syndrome comprises segments for a psychological profile, eye contact, attention deficit disorder (ADD) and attention deficit hyperactivity disorder (ADHD), a social disorder, a range of motor skills, etc. The doctor monitors a patient to determine whether the patient has difficulty in making eye contact, has difficulty interacting socially, lacks interest in spontaneously sharing interests, tends to rely on family or a spouse for all social needs, etc., and records the results of the examination in the condition based stem cell treatment checklist rendered, for example, on an electronic device such as a tablet computing device, a smart phone, etc. The recorded micro-movement data collected by the three-dimensional (3D) motion sensors is tabulated and summarized 104 on a time basis, for example, by day, week, and month. In an embodiment, the recorded micro-movement data is graphed for generating a stem cell treatment plan that defines the dosage of stem cells to be administered to the target object. In an embodiment, the recorded micro-movement data is recorded and tabulated manually. In another embodiment, the recorded micro-movement data is recorded on an electronic device, for example, a tablet computing device or a recorder. The micro-movement data tabulated allows a medical practitioner to adapt 105 the stem cell treatment by adapting a dosage and locations of administration of stem cells in the target object for improved care.

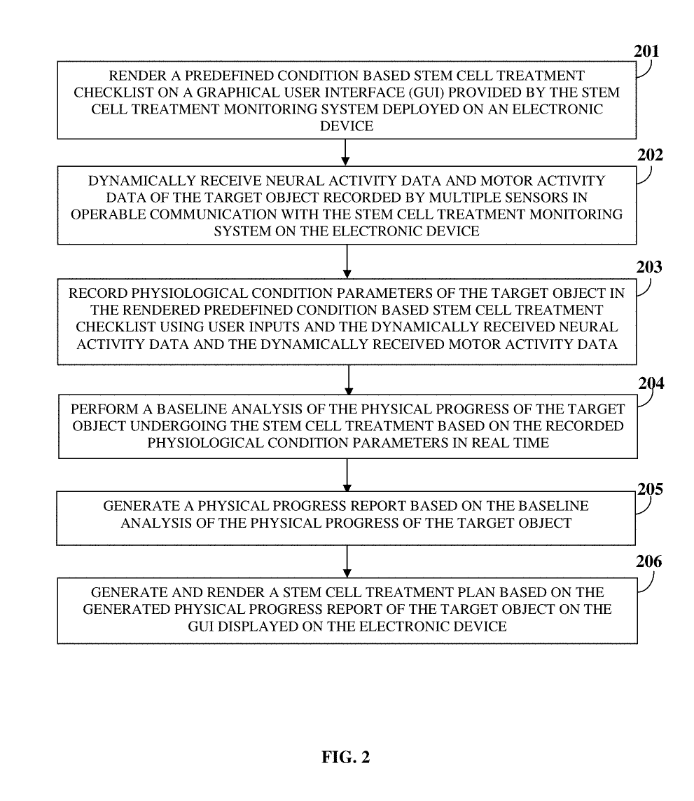

FIG. 2 illustrates an embodiment of the method for monitoring physical progress of a target object undergoing stem cell treatment for determining effectiveness of the stem cell treatment and adapting the stem cell treatment. The method disclosed herein employs a stem cell treatment monitoring system (SCTMS) executable by at least one processor configured to execute computer program instructions for monitoring physical progress of a target object undergoing stem cell treatment for determining the effectiveness of the stem cell treatment and adapting the stem cell treatment. In an embodiment, the SCTMS is a software application such as a mobile application downloadable and usable on an electronic device, for example, a tablet computing device, a smartphone, etc. In another embodiment, the SCTMS is implemented as a web based platform, for example, a website hosted on a server or a network of servers accessible by the electronic device via a network, for example, the internet, a wireless network, a mobile telecommunication network, etc. In another embodiment, the SCTMS is implemented in a cloud computing environment. As used herein, “cloud computing environment” refers to a processing environment comprising configurable computing physical and logical resources, for example, networks, servers, storage, applications, services, etc., and data distributed over a network, for example, the internet. The cloud computing environment provides on-demand network access to a shared pool of the configurable computing physical and logical resources. In an embodiment, the SCTMS is a cloud computing based platform implemented as a service for monitoring physical progress of a target object undergoing stem cell treatment for determining effectiveness of the stem cell treatment and adapting the stem cell treatment. In an embodiment, the SCTMS is developed, for example, using the Google App engine cloud infrastructure of Google Inc., Amazon Web Services® of Amazon Technologies, Inc., the Amazon elastic compute cloud EC2® web service of Amazon Technologies, Inc., the Google® Cloud platform of Google Inc., the Microsoft® Cloud platform of Microsoft Corporation, etc.

In the method disclosed herein, condition based stem cell treatment checklists that define a set of guidelines to be checked or examined for evaluating or diagnosing medical conditions of the target object are provided for ensuring that a medical practitioner, for example, a physician, a doctor, a caregiver, etc., does not miss a diagnosis, and for monitoring ongoing physical progress of the target object while the target object undergoes the stem cell treatment. In an embodiment, the condition based stem cell treatment checklists comprise symptoms, tasks, trackers, indicators of medical conditions, etc., to be monitored or recorded by a physician in accordance with a time schedule for multiple conditions such as brain development, brain injury, brain damage, spinal injury, spinal damage, Lyme disease, Autism, Asperger Syndrome, etc. The stem cell treatment monitoring system (SCTMS) renders 201 a predefined condition based stem cell treatment checklist on a graphical user interface (GUI) provided by the SCTMS deployed on an electronic device to enable a medical practitioner, hereafter exemplarily referred to as a “physician”, to measure and record physiological condition parameters of the target object. As used herein, “physiological condition parameters” refer to physical parameters and/or psychological parameters that identify and determine the medical condition of the target object. The predefined stem cell treatment checklist outlines criteria related to, for example, on of brain development, Lyme disease, a brain injury, a spinal injury, etc.

The stem cell treatment monitoring system (SCTMS) dynamically receives 202 neural activity data and motor activity data of the target object recorded by multiple sensors that are in operable communication with the SCTMS on the electronic device. As used herein, “neural activity data” refers to data related to central nervous system activity and/or fluid flow related to neural activity in a neural system of the target object. Also, as used herein, “fluid flow related to neural activity” refers to a measure of localized changes in cerebral blood flow and/or cerebrospinal fluid flow related to neural activity in the neural system. The neural activity data comprises a change of a fluid flow and an ultra-micro measurement of nerve activity in the neural system of the target object. Also, as used herein, “motor activity data” comprises data on micro-movements of different body parts of the body of the target object as disclosed in the detailed description of FIG. 1.

The sensors are operably coupled to the stem cell treatment monitoring system (SCTMS) on the electronic device and communicate with the SCTMS, for example, via a wired connection, a wireless connection, a network such as a communication network that implements Bluetooth® of Bluetooth Sig, Inc., an ultra-wideband communication network (UWB), a wireless universal serial bus (USB) communication network, a communication network that implements ZigBee® of ZigBee Alliance Corporation, etc. The sensors comprise neural sensors positioned proximal to the neural system of the target object for measuring the neural activity data comprising a change of fluid flow and an ultra-micro measurement of nerve activity in the neural system of the target object. In an embodiment, the neural sensors comprise a laser Doppler flow meter and electrodes configured to measure the neural activity data comprising information of a blood cell perfusion in microvasculature and the ultra-micro measurement of the nerve activity in a spine and a brain of the target object. The laser Doppler flow meter is, for example, the Blood FlowMeter of ADInstruments Pty. Ltd., Unit 13, 22 Lexington Drive Bella Vista New South Wales, Australia 2153. The laser Doppler flow meter focuses a laser beam into a small volume of a flowing fluid containing small particles. The small particles scatter the laser beam with a Doppler shift. Magnitude and frequency distribution analysis of the scattered laser beam accurately determines neural activity and velocity of the small particles. For example, the laser Doppler flow meter is positioned proximal to a volume of tissue of the brain of the target object to measure a blood cell perfusion in the microvasculature of the brain of the target object undergoing the stem cell treatment. The laser Doppler flow meter emits a low power laser light on the measuring volume of tissue of the brain. The emitted low power laser light is backscattered in dynamic red blood cells and in static tissues of the measuring volume of tissue of the brain of the target object. The magnitude and frequency distribution of the backscattered light relates to the neural activity and the velocity of moving blood cells within the measuring volume of tissue of the brain of the target object undergoing the stem cell treatment. Example laser Doppler flow meter readings of cerebral blood flow (CBF) are provided in Table 1 below.

TABLE 1

| Blood pressure, | | Left CBF, | | Right CBF, | |
|---|---|---|---|---|---|
| mmHg | | mL/100 g/min | | mL/100 g/min | |
| Δ Mean | Δ Cerebral | | CBF | | CBF |
| Arterial | perfusion | CBF baseline | absolute | CBF baseline | absolute |
| Pressure | pressure | value | value | value | value |
| −35 | −35 | 53.5 | 32.2 | 63.2 | 45.1 |
| −7 | −9 | 57.6 | 54.2 | 47.0 | 46.5 |
| 25 | 23 | 38.5 | 25.9 | 18.3 | 27.7 |

The sensors of the stem cell treatment monitoring system (SCTMS) further comprise the wearable motion monitoring device configured, for example, as a full body suit that conforms to the body of the target object. The wearable motion monitoring device comprises motion sensors for measuring the motor activity data comprising data on micro-movements of one or more different body parts of the body of the target object. The micro-movements define a range of motion for fine motor skills or gross motor skills of the target object. The target object wears the wearable motion monitoring device to allow the motion sensors to measure the micro-movements of the different body parts of the body of the target object undergoing the stem cell treatment. The motion sensors in the wearable motion monitoring device are positioned on different body parts of the target object, for example, ankle, thigh, wrist, waist, chest, head, etc. The wearable motion monitoring system measures the micro-movements of the target object using the motion sensors positioned on different body parts of the target object undergoing the stem cell treatment. A generic computer using a generic program cannot dynamically receive neural activity data and motor activity data of the target object from multiple sensors that are in operable communication with the SCTMS in accordance with the method steps disclosed above.

The stem cell treatment monitoring system (SCTMS) records 203 physiological condition parameters of the target object in the rendered predefined condition based stem cell treatment checklist using user inputs and the dynamically received neural activity data and the dynamically received motor activity data. The user inputs comprise, for example, inputs from a physician on the predefined condition based stem cell treatment checklist rendered on the graphical user interface (GUI) displayed on the physician's electronic device, on performing an examination or an initial assessment on the target object. In an embodiment, the physiological condition parameters are input into the predefined condition based stem cell treatment checklist on the GUI displayed on the physician's electronic device. In an example, a spinal cord injury (SCI) based stem cell treatment checklist comprises a set of guidelines to be checked or examined for evaluating and/or diagnosing a spinal cord injury of the patient. The SCI based stem cell treatment checklist comprises, for example, cause of the patient's spinal cord injury, whether the patient has sensation below the level of spinal cord injury, whether the patient is not able to sit, whether the patient has impaired mobility and/or sensation below the level of the spinal cord injury, whether the patient maintains some sensation and/or mobility below the level of the spinal cord injury or under a relatively lower level of the spinal cord injury, etc. The physician assesses the physiological condition parameters associated with the spinal cord injury of the patient undergoing the stem cell treatment using the SCI based stem cell treatment checklist and records the physiological condition parameters into the SCI based stem cell treatment checklist rendered on the GUI on the electronic device. For example, the physician inputs the cause of the patient's spinal cord injury and then, using the neural activity data and the motor activity data dynamically received from the neural sensors and the motion sensors respectively, checks or unchecks checkboxes positioned adjacent to the guidelines in the SCI based stem cell treatment checklist to indicate whether the patient has sensation below the level of spinal cord injury, whether the patient is not able to sit, whether the patient has impaired mobility and/or sensation below the level of the spinal cord injury, whether the patient maintains some sensation and/or mobility below the level of the spinal cord injury or under a relatively lower level of the spinal cord injury, etc., via the GUI. The data inputted by the physician in the predefined condition based stem cell treatment checklist via the GUI is transformed, processed, and executed by an algorithm in the SCTMS to record the physiological condition parameters of the target object undergoing the stem cell treatment.

The physician selects the predefined condition based stem cell treatment checklist on the graphical user interface (GUI) displayed on the electronic device based on the medical condition of the target object. The stem cell treatment monitoring system (SCTMS) renders the selected predefined condition based stem cell treatment checklist based on the medical condition of the target object on the GUI displayed on the electronic device. The SCTMS provides input interface elements comprising, for example, checkboxes, slide bars, push buttons, etc., for the physician to monitor and record the physiological condition parameters of the target object on the predefined condition based stem cell treatment checklist rendered on the GUI. The SCTMS analyzes the physiological condition parameters of the target object recorded in each segment of the predefined condition based stem cell treatment checklist. The SCTMS evaluates symptoms and severity of the medical condition of the target object based on the recorded physiological condition parameters and establishes a diagnostic score associated with the medical condition. In the above example of a patient undergoing stem cell treatment for a spinal cord injury, the SCTMS analyzes the physiological condition parameters inputted by the physician and establishes diagnostic scores for the spinal cord injury of the patient as follows:

0—Lack of response from muscles
1—Involuntary, unsustained muscle movement, that is, tremors
2—Muscle movement in gravity eliminated condition
3—Muscle movement against gravity
4—Muscle movement against gravity with some resistance
5—Muscle movement against gravity with some resistance

The stem cell treatment monitoring system (SCTMS) performs 204 a baseline analysis of the physical progress of the target object undergoing the stem cell treatment based on the recorded physiological condition parameters in real time. As used herein, “baseline” refers to a line that is a base for measurement of the physical progress of the target object. Also, as used herein, “baseline analysis” refers to a process of establishing an initial assessment against which the physical progress of the target undergoing the stem cell treatment is monitored and measured. The baseline analysis provides information on the medical condition of the target object that the stem cell treatment aims to change. The baseline analysis provides a reference point for assessing changes in the medical condition and impact of the stem cell treatment, as the baseline analysis establishes a basis for comparing the medical condition of the target object before and after the stem cell treatment and for inferring the effectiveness of the stem cell treatment. The physiological condition parameters recorded using the neural activity data and the motor activity data dynamically received from the sensors provide a baseline understanding of motion, for example, gross motor skills such as range of limb movement, etc., fine motor skills such as hand-eye coordination, etc.

In an embodiment, the stem cell treatment monitoring system (SCTMS) receives one or more color coded tomographic images and supplementary data of the target object from one or more external sources. As used herein “color coded tomographic images” refer to functional images, for example, single-photon emission computed tomography (SPECT) images and positron emission tomography (PET) images of a body part, for example, brain, spine, etc., of the target object. Also, as used herein, “functional images” refer to three-dimensional cross-sectional images indicating localized changes in fluid flow and activity of a body part, for example, the brain of the target object. The localized changes in fluid flow and activity are represented by multiple colors in the color coded tomographic images. The colors illustrate a change of fluid flow and regions of the body part of the target object that are activated when the target object performs a particular task. For example, the SPECT images and/or the PET images of the brain of the target object illustrate widespread activation of the frontal lobe of the brain of the target object with a predetermined color when the target object performs a task that involves motor activity. Also, as used herein, “supplementary data” refers to healthcare data of the target object, for example, medical reports containing medical history of the target object, medical records, and physical therapy records of the target object. The color coded tomographic images and the supplementary data of the target object received by the SCTMS are transformed, processed, and executed by an algorithm in the SCTMS for performing a baseline analysis of the physical progress of the target object undergoing the stem cell treatment.

In an embodiment, the stem cell treatment monitoring system (SCTMS) correlates the received color coded tomographic images and the received supplementary data with the recorded physiological condition parameters to perform the baseline analysis of the physical progress of the target object undergoing the stem cell treatment. The color coded tomographic images of the body part of the target object received by the SCTMS are analyzed, for example, using an object based region of interest (ROI) technique and a functional imaging analysis (FMA) technique. As used herein, “object based ROI” refers to a region containing user defined objects of interest. The object based ROI technique utilizes a discriminant saliency principle to discriminate the objects of interest from others by defining image regions of strongest response as salient. The discriminant saliency principle comprises a saliency detection stage for detecting salient points present in an image and a saliency validation stage for verifying consistency of a geometric configuration of salient points in the image. For example, the SCTMS receives single-photon emission computed tomography (SPECT) images and/or the positron emission tomography (PET) images of the brain of a patient undergoing stem cell treatment for brain injury. The SCTMS analyzes the SPECT images and/or PET images of the patient's brain using the object based ROI techniques. The SCTMS identifies cerebral perfusions and/or vascular lesions present in the SPECT images and/or the PET images of the brain using the saliency detection stage. The SCTMS locates the region and/or boundary of the cerebral perfusions and/or the vascular lesions using the saliency validation stage.

The functional imaging analysis technique refers to a method for detecting and/or measuring changes in physiological activities, for example, metabolism, blood flow, regional chemical composition, etc., within a particular body part of the target object based on an analysis of images. The stem cell treatment monitoring system (SCTMS) uses the functional imaging analysis technique to analyze the color coded tomographic images. The SCTMS using the functional imaging analysis technique accurately identifies and/or locates perfusion abnormalities resulting from primary vascular problems or from neuronal dysfunction in the body part of the target object undergoing stem cell treatment. The SCTMS establishes a correlation between the identified perfusion abnormalities with the neuropsychological conditions or neuropsychological deficits. In an example, the SCTMS receives single-photon emission computed tomography (SPECT) images and/or the positron emission tomography (PET) images of the brain of a patient undergoing stem cell treatment for brain injury. The SCTMS analyzes the SPECT images and/or PET images using the functional imaging analysis techniques to identify and correlate a decreased blood flow to various parts of the brain with various types of behavior, for example, frontal lobes with disinhibitive behavior, left cerebral hemisphere with increased social isolation, right hemispheric areas with increased aggressive behavior, deficits in frontal lobe and thalamic perfusion indicate impairments in executive functioning, etc.

During the baseline analysis, the stem cell treatment monitoring system (SCTMS) analyzes the recorded physiological condition parameters that define the medical condition of the target object corresponding to the predefined condition based stem cell treatment checklist along with the received color coded tomographic images and the supplementary data, and then generates a baseline assessment. For example, the SCTMS generates an Asperger syndrome assessment related to a psychological profile, eye contact, attention deficit, social disorder, range of motor skills, etc., for the target object. A generic computer using a generic program cannot perform a baseline analysis of the physical progress of the target object undergoing the stem cell treatment based on the recorded physiological condition parameters in real time in accordance with the method steps disclosed above.

In an embodiment, the stem cell treatment monitoring system (SCTMS) processes and transforms the recorded physiological condition parameters, the dynamically received neural activity data, and the dynamically received motor activity data to generate target object activity information. As used herein, “target object activity information” refers to structured information in a predefined format that illustrates, for example, changes in the neural activity with respect to changes in the motor activity of the target object undergoing the stem cell treatment. The target object activity information comprises, for example, information on gross motor skills, fine motor skills, fluid flow in a brain of the target object, etc. The SCTMS dynamically receives neural activity data shown in Table 1 above and motor activity data measured by the sensors connected to the target object. The SCTMS processes and identifies relevant data from the motor activity data and the neural activity data based on a medical condition of the target object. The SCTMS extracts the identified relevant data by using, for example, feature extraction and dimension reduction techniques. The SCTMS classifies the extracted data using a dimensionally reduced feature to generate the target object activity information. The SCTMS renders the generated target object activity information on the graphical user interface (GUI) displayed on the electronic device. The SCTMS stores the generated target object activity information in an information database. A generic computer using a generic program cannot generate the target object activity information by processing and transforming the dynamically received neural activity data and the dynamically received motor activity data in accordance with the method steps disclosed above. The dynamically received neural activity data and the dynamically received motor activity data are transformed, processed, and executed by an algorithm in the SCTMS for generating the target object activity information.

The stem cell treatment monitoring system (SCTMS) generates 205 a physical progress report based on the baseline analysis of the physical progress of the target object for determining the effectiveness of the stem cell treatment. The physical progress report is an electronic document comprising information and output results of the baseline analysis performed by the SCTMS and the physical progress of the target object undergoing the stem cell treatment. The SCTMS renders the generated physical progress report of the target object on the graphical user interface (GUI) displayed on the electronic device. The generated physical progress report is stored in the information database. The physical progress report enables a physician to determine the effectiveness of the stem cell treatment, determine type of stem cells to be administered, dosage of the stem cells to be administered, and duration for the next base line analysis of the target object undergoing the stem cell treatment. A generic computer using a generic program cannot generate and render the physical progress report based on the baseline analysis of the physical progress of the target object on the GUI displayed on the electronic device for determining the effectiveness of the stem cell treatment in accordance with the method steps disclosed above.

The stem cell treatment monitoring system (SCTMS) generates and renders 206 a stem cell treatment plan based on the generated physical progress report of the target object on the graphical user interface (GUI) displayed on the electronic device. The generated stem cell treatment plan comprises, for example, information on the type of stem cells to be administered to the target object, dosage of the stem cells to be administered to the target object, duration for the next baseline analysis, area of administration of the stem cells such as neck, spine, other areas of the body of the target object, physical therapy and behavioral therapy to be administered to the target object, etc. In an example of a patient suffering from Lyme disease, although the Lyme disease does not directly affect a patient's physical range of motion and strength, the Lyme disease does create fatigue. Alongside with the stem cell treatment, the SCTMS generates a stem cell treatment plan comprising physiotherapy aimed to increase the patient's endurance and build stamina. In an embodiment, the SCTMS analyzes the physical progress report of the target object and communicates with external data sources and entities, for example, stem cell experts via a network, for example, the internet to generate an optimal stem cell treatment plan. A generic computer using a generic program cannot generate a stem cell treatment plan based on the generated physical progress report in accordance with the method steps disclosed above. The generated stem cell treatment plan allows the physician to adapt the stem cell treatment, for example, by adapting a dosage and locations of administration of stem cells in the target object for improved care.

The stem cell treatment monitoring system (SCTMS) enables a physician to monitor physical progress of the target object undergoing the stem cell treatment and determine effectiveness of the stem cell treatment using the electronic device. On implementing the method disclosed herein, the end result generated by the SCTMS is a tangible determination of physical progress of the target object undergoing the stem cell treatment and a tangible determination of the effectiveness of the stem cell treatment.

Consider an example where a patient, aged 36, suffering from quadriplegia due to a spinal cord injury (SCI) is admitted for stem cell treatment. With a spinal cord injury, the patient has impaired mobility and sensation below the level of the spinal cord injury. Before the stem cell treatment, the patient initially had no sensation below the level of the spinal cord injury, was not able to sit and stand, and had no movements below the lower limbs. The doctor treats the patient by injecting stem cells close to the spinal cord to allow the stem cells to regenerate an injured area near the spinal cord. The doctor injects a catheter in the spine and periodically feeds the stem cells through the catheter. The catheter is strategically located as close to the level of injury as possible so that the stem cells have less of a distance to travel. The doctor activates the stem cell treatment monitoring system (SCTMS) on an electronic device, for example, a tablet computing device and initiates a search for an SCI based stem cell treatment checklist on the tablet computing device. The SCTMS renders the SCI based stem cell treatment checklist disclosed above on the graphical user interface (GUI) displayed on the tablet computing device. The doctor instructs the patient to wear the wearable motion monitoring device configured as a full body suit. The motion sensors in the wearable motion monitoring device measure the motor activity data comprising data on micro-movements of muscles, upper limb, elbow, wrist, fingers, and hips of the patient. The motion sensor of the wearable motion monitoring device connected to the upper limb of the patient measures motor activity comprising, for example, movement of flexors on the right side, flexors on the left side, extensors on the right side, extensors on the left side, abductors on the right side, abductors on the left side, external rotator on the right side, external rotator on the left side, internal rotator on the right side, internal rotator on the left side, etc., of the upper limb of the patient. Similarly, the motion sensors of the wearable motion monitoring device connected to the elbow, the wrist, the fingers, the hips, etc., measure motor activity of the elbow, the wrist, the fingers, the hips, etc., respectively. The doctor, using the SCI stem cell treatment checklist, initiates a patient diagnosis to assess physiological condition parameters that indicate whether the patient has sensation below the level of injury, whether the patient is able to sit, whether the patient is able to stand, whether the patient can move below lower limbs, etc. The doctor records the patient's physiological condition parameters using the doctor's inputs and the motor activity data received from the sensors, along with notes comprising, for example, a superficial sensation, pain till abdomen below the level of injury, light touch till abdomen below the level of injury, deep sensation present till abdomen below the level of injury, etc., in the SCI stem cell treatment checklist displayed on GUI of the tablet computing device.

The stem cell treatment monitoring system (SCTMS) performs a baseline analysis of the physical progress of the patient based on the recorded physiological condition parameters as disclosed above. The SCTMS generates a physical progress report comprising, for example, movement of flexors on the right side improved from 1-3, movement of flexors on the left side improved from 1-3, movement of extensors on the right side improved from 0-1, movement of the extensors on the left side improved from 0-1, movement of abductors on the right side stayed at 4, movement of abductors on the left side stayed at 3, movement of the external rotator on the right side stayed at 4, movement of the external rotator on the left side stayed at 3, movement of the internal rotator on the right side stayed at 2, movement of the internal rotator on the left side stayed at 2, etc., based on the baseline analysis of the patient and renders the generated physical progress report on the graphical user interface (GUI) displayed on the tablet computing device to the doctor. The physical progress report allows the doctor to determine the effectiveness of the stem cell treatment. The SCTMS analyzes the generated physical progress report to generate a stem cell treatment plan comprising, for example, type of stem cells to be administered to the patient, dosage of the stem cells to be administered to the patient, area of administration of the stem cells to improve overall muscle strength, improve sitting balance, improve endurance, allow the patient to start standing and walking, correct deformities. The SCTMS determines the duration, for example, one month for the next baseline analysis.

Consider another example where a patient suffering from the Asperger syndrome, also referred to as Asperger's, undergoes stem cell treatment. The doctor activates the stem cell treatment monitoring system (SCTMS) on an electronic device, for example, a tablet computing device and initiates a search for a stem cell treatment checklist based on Asperger's on the tablet computing device. The SCTMS renders the stem cell treatment checklist based on Asperger's comprising segments for a psychological profile, an attention deficit disorder, eye contact, social disorder, and range of motor skills on the graphical user interface (GUI) displayed on the tablet computing device. The doctor instructs the patient to wear the wearable motion monitoring device configured as a full body suit. The motion sensors in the wearable motion monitoring device connected to the patient's hands measure the motor activity data comprising data on micro-movements of the hands, fingers, etc., of the patient. The motion sensors in the wearable motion monitoring device connected to the patient's hands measure motor activity comprising movement of fingers, handwriting skills, difficulty with balance, etc., of the patient's hands. The doctor, using the stem cell treatment checklist based on Asperger's, initiates a patient diagnosis for attention deficit disorder by checking whether the patient is often easily distracted, whether the patient is often forgetful in daily activities, how often the patient does not seem to listen when spoken to directly, etc. The doctor records the patient's physiological condition parameters by providing inputs to the segments on the stem cell treatment checklist based on Asperger's displayed on the GUI on the tablet computing device using the doctor's evaluation inputs and the motor activity data received from the sensors. The motor activity data provides information on whether the patient maintains eye contact, the patient's hand eye coordination, handwriting skills, imitating gestures, etc.

The stem cell treatment monitoring system (SCTMS) performs a baseline analysis of the physical progress of the patient based on the recorded physiological condition parameters as disclosed above. The SCTMS generates a physical progress report comprising, for example, impaired two way social interaction, lack of facial expression, poor hand to eye coordination, etc., based on the baseline analysis of the patient and renders the generated physical progress report on the graphical user interface (GUI) displayed on the tablet computing device to the doctor. The SCTMS analyzes the generated physical progress report to generate a stem cell treatment plan comprising, for example, behavioral therapy, physical therapy, type of stem cells to be administered to the patient, dosage of the stem cells to be administered to the patient, area of administration of the stem cells to improve overall. The SCTMS also determines the duration, for example, three months for the next baseline analysis.

Consider another example where a doctor using a tablet computing device with the stem cell treatment monitoring system (SCTMS) installed thereon initiates monitoring of physical progress of a patient by performing a patient diagnosis based on a condition based stem cell treatment checklist for conditions such as brain development issues such as autism spectrum disorder, attention deficit disorder, or a brain injury, or a spinal injury, or Lyme disease. The doctor collects information through a conversation diagnosis directed by key points indicated in the condition based stem cell treatment checklist displayed on the graphical user interface (GUI) on the tablet computing device. This allows the doctor to perform a detailed examination of the patient. The doctor inputs the collected information into the condition based stem cell treatment checklist displayed on the GUI on the tablet computing device. Scans, for example, brain scans or single-photon emission computed tomography (SPECT) images for areas such as the brain and other damaged areas such as the spine or other bodily areas are collected for examination and inputted or transmitted to the SCTMS on the electronic device. Supplementary data, for example, prior patient records can also be stored or accessed from the information database of the SCTMS.

The doctor then measures a baseline for motion. The patient wears the wearable motion monitoring device configured, for example, as a full body motion monitoring body suit and the doctor attaches the neural sensors to the patient to collect information to be fed into the tablet computing device. The stem cell treatment monitoring system (SCTMS), in combination with the neural sensors and the wearable motion monitoring device, measures micro-movements for both fine and gross motor skill management and records a real movement baseline for the patient. After measurement of motion, the SCTMS records other baseline patient conditions by type of alignment using the sensors. As an example, a patient suffering from an Asperger Syndrome condition is evaluated by a psychological profile and a degree of eye contact during conversation, and checked for conditions of attention deficit disorder and other social disorders.

Based on the information collected, the stem cell treatment monitoring system (SCTMS) generates a physical progress report. The doctor, assisted by supplementary data comprising, for example, heuristics on the tablet computing device, and the physical progress report displayed on the graphical user interface (GUI) on the electronic device, generates an individualized stem cell treatment plan comprising, for example, type of cells, duration of assessment, location and therapy program both physical and behavioral to be administered to the patient, etc., for the patient.

The doctor performs an ongoing patient assessment as follows: Based on the individualized stem cell treatment plan defined for the patient, all aspects of the patient activity are measured through the sensors and the stem cell treatment monitoring system (SCTMS) on an ongoing basis. The doctor provides the patient daily stem cell injections, physical therapy, and behavioral therapy. The doctor inputs a record of the daily stem cell treatment into the SCTMS on the tablet computing device and conducts physical therapy sessions by performing motion and behavioral monitoring on the patient wearing the full body motion monitoring body suit for gross motor skills and attaches sensors to the patient's hand for writing and other fine motor skills measurement recording. The SCTMS generates a weekly physical progress report for the doctor who can then review and request modifications to all types of stem cell treatments. The SCTMS also generates a monthly physical progress report comprising summarized progress of the patient and scans and required X-rays.

Based on the physical progress of the patient, the doctor can suggest a return post real world activity or close out the patient through the stem cell treatment monitoring system (SCTMS) by performing before and after reporting for all scanning, changes observed, and reviews of video, single-photon emission computed tomography (SPECT) scans, or spinal activity highlighting how the patient has progressed. The SCTMS close out physical progress reports that show the changed range of motion for fine and gross motor skills and behavioral changes. As the stems cells continue to increase and provide improvement, the SCTMS generates a stem cell treatment plan for patient activity to be conducted post departure from facilities.

The stem cell treatment monitoring system (SCTMS) disclosed herein improves the functionality of the electronic device, for example, a tablet computing device, a laptop, a smartphone, etc., and provides an improvement in computer related technology as follows: The SCTMS generates and renders the condition based stem cell treatment checklist relevant to the medical condition of the target object on the graphical user interface (GUI) on the electronic device to enable the physician to measure and record the physiological condition parameters of the target object. To generate the condition based stem cell treatment checklist, the SCTMS integrates multiple aspects and guidelines relevant to the stem cell treatment of the medical condition from a medical database. A generic computer using a generic program cannot generate a condition based stem cell treatment checklist by integrating guidelines relevant to the medical condition from the medical database in accordance with the method steps disclosed above.

The stem cell treatment monitoring system (SCTMS) provides multiple sensors to monitor and measure the neural activity and the motor activity of the target object undergoing stem cell treatment. The SCTMS correlates the dynamically measured neural activity and the measured motor activity to generate target object activity information in real time. The generated target object activity information illustrates the change in the neural activity in response to corresponding motor activity performed by the target object. For example, the target object activity information illustrates widespread activation of the occipital lobe of the brain of the target object when the target object performs a task that involves visual activity. Furthermore, the SCTMS correlates the physiological condition parameters, the color coded tomographic images, the supplementary data, and the target object activity information of the target object to establish a baseline analysis of the target object in real time. The SCTMS generates the physical progress report of the target object undergoing the stem cell treatment using the established baseline analysis in real time. The end result of generating the physical progress report to monitor physical progress of the target object undergoing stem cell treatment and to determine effectiveness of the stem cell treatment is tangible and not abstract.

In the method disclosed herein, the design and flow of interactions in the system disclosed herein between the stem cell treatment monitoring system (SCTMS) installed on the electronic device, the neural sensors and the motion sensors of the wearable motion monitoring device connected to the target object undergoing the stem cell treatment, and the external sources are deliberate, designed, and directed. Every prompt, every guideline, etc., the physician receives via the graphical user interface (GUI) is configured by the SCTMS to enable the physician to monitor, record, and determine effectiveness of the stem cell treatment. The SCTMS implements one or more specific computer programs to direct the physician towards a set of end results. The interactions designed by the system allow the SCTMS to collect information comprising the physiological condition parameters, the color coded tomographic images, the supplementary data, and the target activity information on the electronic device. From this information, the SCTMS, through the use of another, separate and autonomous computer program, performs the baseline analysis of the target object undergoing stem cell treatment in real time. The established baseline analysis is used by the SCTMS to generate and render the physical progress report of the target object undergoing stem cell treatment on the electronic device in real time. To dynamically measure the motor activity data and the neural activity data using multiple sensors, record the physiological condition parameters, generate target object activity information in real time, perform a baseline analysis, generate and display a physical progress report on the electronic device, and generate and display a stem cell treatment plan on the electronic device in real time requires six or more separate computer programs, the execution of which cannot be performed by a person using a generic computer with a generic program. The method disclosed herein provides an advancement to stem cell treatment using the advancement of the sensors and a combination of technologies and a repeatable process on an electronic device with correlated data that provides medical practitioners, for example, doctors a method to monitor the physical progress of the target object and change the stem cell treatment with revised stem cell strategies.

The focus of the method disclosed herein is on an improvement to stem cell treatment using a combination of the sensors and the stem cell treatment monitoring system (SCTMS) installed on the electronic device, and not on other tasks for which a generic computer is used in its ordinary capacity. Accordingly, the method and the system disclosed herein are not directed to an abstract idea. Rather, the method and the system disclosed herein are directed to a specific improvement to the way the sensors and the SCTMS on the electronic device operate, embodied in, for example, reception of one or more color coded tomographic images and supplementary data of the target object from one or more external sources, dynamic reception of neural activity data and motor activity data of the target object from the sensors, recording of physiological condition parameters of the target object in the predefined stem cell treatment checklist using user inputs and the dynamically received neural activity data and the dynamically received motor activity data of the target object, generation of target object activity information, performance of the baseline analysis of the physical progress of the target object undergoing the stem cell treatment based on the recorded physiological condition parameters, the received color coded tomographic images, the supplementary data, and the generated target object activity information in real time, generation and rendering of the physical progress report based on the baseline analysis of the physical progress of the target object on the graphical user interface (GUI) provided by the SCTMS on the electronic device for determining the effectiveness of the stem cell treatment, and generation and rendering of the stem cell treatment plan based on the physical progress report on the GUI.

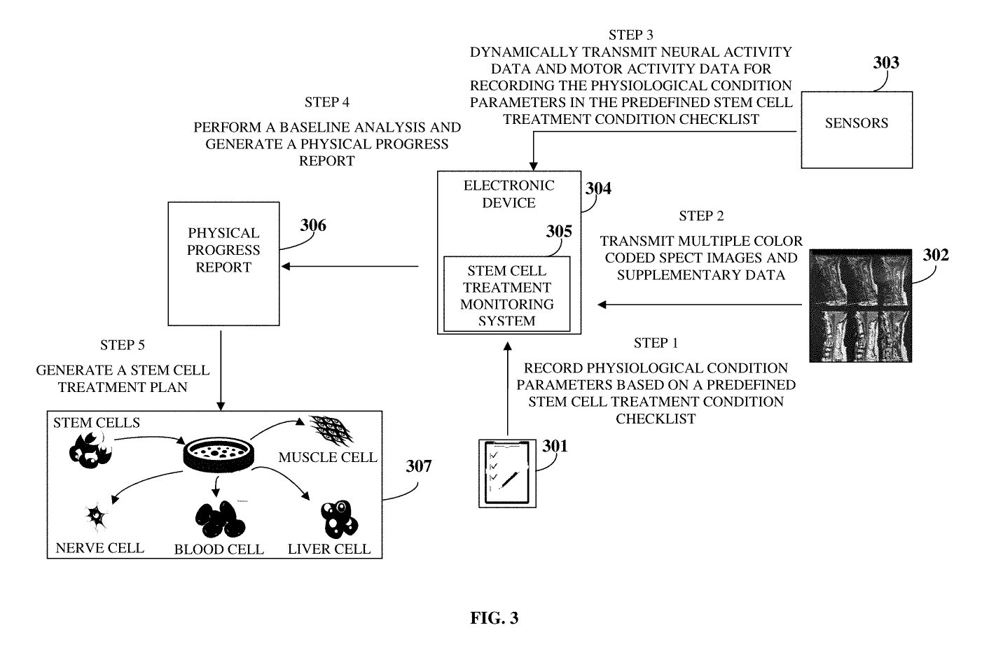

FIG. 3 exemplarily illustrates a schematic representation of the embodiment of the method for monitoring physical progress of a target object undergoing stem cell treatment for determining effectiveness of the stem cell treatment and adapting the stem cell treatment. Consider an example where a physician examines a patient undergoing stem cell treatment for a brain injury using the stem cell treatment monitoring system (SCTMS) 305 deployed on an electronic device 304, for example, a tablet computing device or a smartphone. The SCTMS 305 on the electronic device 304 assists the physician to monitor real time change by viewing a scan on the graphical user interface (GUI) of the SCTMS 305 on the electronic device 304, while focusing on activity that requires improvement and measuring the change. In step 1, the physician records the physiological condition parameters of the patient based on a predefined condition based stem cell treatment checklist 301 for the brain injury rendered by the SCTMS 305 on the electronic device 304 to determine the condition of the patient suffering from the brain injury. In step 2, periodic single-photon emission computed tomography (SPECT) images 302 are taken through a scanner and transmitted to the SCTMS 305 on the electronic device 304 along with supplementary data comprising, for example, the medical reports of the patient. The physician examines the SPECT images 302 of the brain of the patient, received by the SCTMS 305 on the electronic device 304 to evaluate the location and severity of the brain injury of the patient. The SCTMS 305 displays the color coding of brain areas that are strong and weak on the GUI.

The doctor connects the sensors 303 to the patient, for example, by connecting the neural sensors to the patient's head or by requesting the patient to wear the wearable motion monitoring device with the motion sensors, for monitoring and measuring neural activity and motor activity of the patient. In step 3, the sensors 303 dynamically transmit neural activity data and motor activity data to the stem cell treatment monitoring system (SCTMS) 305 on the electronic device 304 for recording the physiological condition parameters of the patient in the predefined stem cell treatment checklist 301. In an embodiment, the SCTMS 305 processes the measured neural activity data and motor activity data from the sensors 303 and generates the target object activity information, herein referred to as “patient activity information”. In step 4, the SCTMS 305 performs a baseline analysis by correlating the recorded physiological condition parameters, the generated patient activity information, and the single-photon emission computed tomography (SPECT) images 302 to establish a baseline assessment of the physical progress of the patient, generates a physical progress report 306 based on the baseline analysis, and displays the generated physical progress report 306 on the graphical user interface (GUI) on the electronic device 304. The doctor analyzes the physical progress report 306 displayed on the electronic device 304. In an embodiment, in step 5, the SCTMS 305 generates a stem cell treatment plan 307 to determine the type of stem cells to be administered to the patient, dosage of the stem cells to be administered to the patient, duration of the baseline analysis, area of administration of the stem cells, and physical therapy and behavioral therapy to be administered to the patient suffering from the brain injury. The SCTMS 305 provides daily patient progress data that allows the doctor to adapt the stem cell treatment.

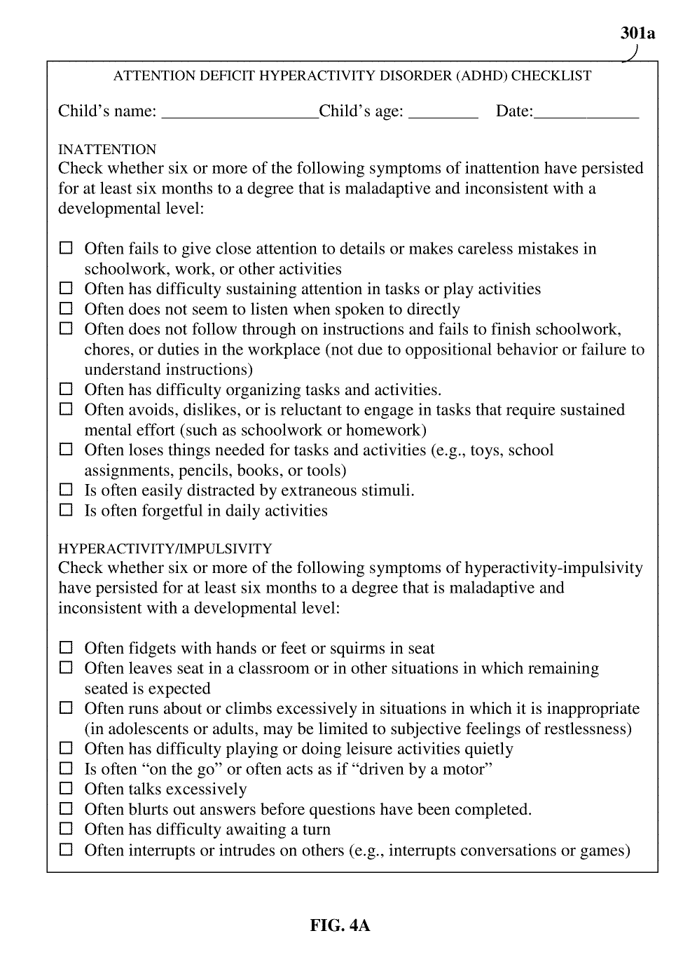

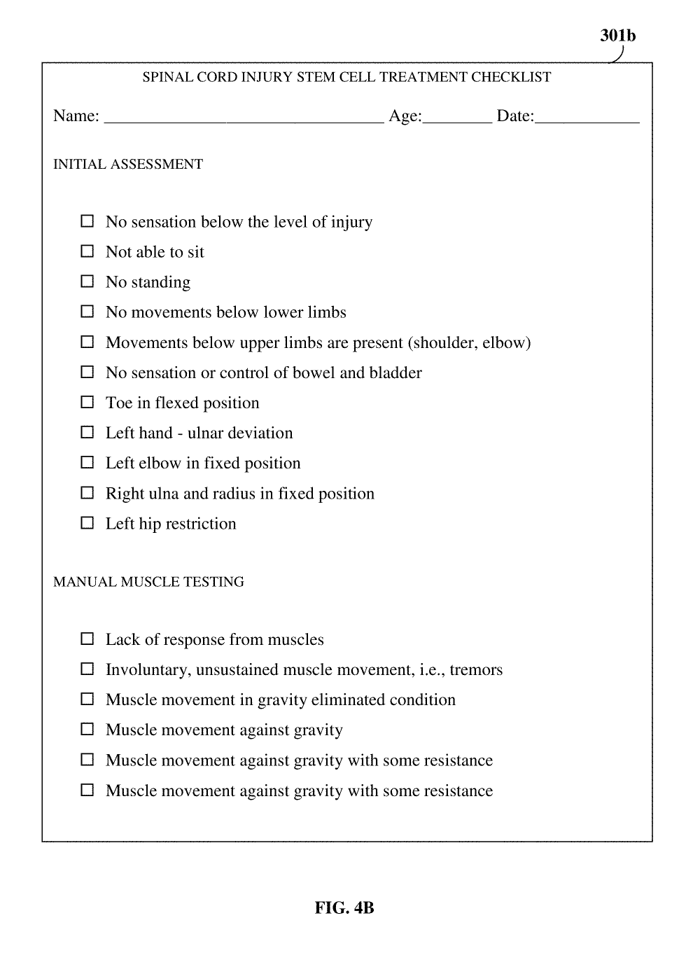

FIGS. 4A-4B exemplarily illustrate predefined condition based stem cell treatment checklists 301a and 301b used for recording physiological condition parameters of a target object undergoing stem cell treatment. A predefined stem cell treatment checklist 301a based on attention deficit hyperactivity disorder (ADHD) is exemplarily illustrated in FIG. 4A. To monitor physical progress of a patient with ADHD undergoing stem cell treatment, the stem cell treatment monitoring system (SCTMS) 305 exemplarily illustrated in FIG. 3, renders the ADHD stem cell treatment checklist 301a on the graphical user interface (GUI) displayed on a doctor's electronic device 304 exemplarily illustrated in FIG. 3. As exemplarily illustrated in FIG. 4A, the ADHD stem cell treatment checklist 301a comprises a list of symptoms of inattention and hyperactivity-impulsivity. The doctor is required to check whether the symptoms of inattention and hyperactivity-impulsivity have persisted for at least six months to a degree that is maladaptive and inconsistent with a developmental level. The ADHD stem cell treatment checklist 301a allows the doctor to record the psychological condition parameters that indicate, for example, how often the patient fails to give close attention to details or makes careless mistakes in schoolwork, work, or other activities, how often the patient has difficulty sustaining attention in tasks or play activities, how often the patient fidgets with hands or feet or squirms in a seat, how often the patient runs about or climbs excessively in situations in which it is inappropriate, etc.

A predefined stem cell treatment checklist 301b based on a spinal cord injury (SCI) is exemplarily illustrated in FIG. 4B. To monitor physical progress of a patient with a spinal cord injury undergoing stem cell treatment, the stem cell treatment monitoring system (SCTMS) 305 renders the SCI stem cell treatment checklist 301b on the graphical user interface (GUI) displayed on the doctor's electronic device 304. As exemplarily illustrated in FIG. 4B, the SCI stem cell treatment checklist 301b comprises a list of symptoms of a spinal cord injury. The SCI stem cell treatment checklist 301b allows the doctor to record the psychological condition parameters that indicate, for example, whether the patient has sensation below the level of injury, whether the patient is able to sit, whether the patient is able to stand, whether there are movements below the lower limbs, whether there are movements below the upper limbs, etc. The doctor records the psychological condition parameters using the motor activity data measured by the motion sensors in the wearable motion monitoring device worn as a full body suit by the patient.

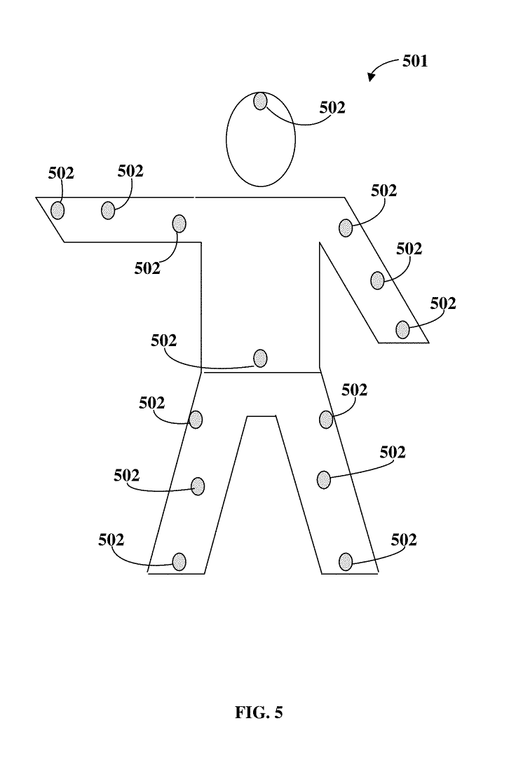

FIG. 5 exemplarily illustrates the wearable motion monitoring device 501 configured as a full body suit comprising multiple motion sensors 502 positioned on locations corresponding to different body parts, for example, the head, arms, legs, torso, etc., of a body of the target object. The motion sensors 502 comprise one of microelectromechanical system (MEMS) sensors, accelerometers, inertial sensors, gyroscopes, etc., or any combination thereof. The MEMS sensors facilitate kinematic measurements. In an embodiment, high speed, or optical and/or optoelectronic cameras are used to generate kinematics needed for analysis. In another embodiment, force plates and/or electromyography is combined with the motion sensors 502 to provide complete information to the stem cell treatment monitoring system (SCTMS) 305 exemplarily illustrated in FIG. 3. The motion sensors 502 dynamically measure the micro-movements of the body part on which the motion sensors 502 are positioned to collect angular velocity data, acceleration data, position data, and orientation data related to the respective body parts of the target object. The measurements of the motion sensors 502 are used to generate the motor activity data of the target object undergoing stem cell treatment as disclosed in detailed description of FIG. 2.

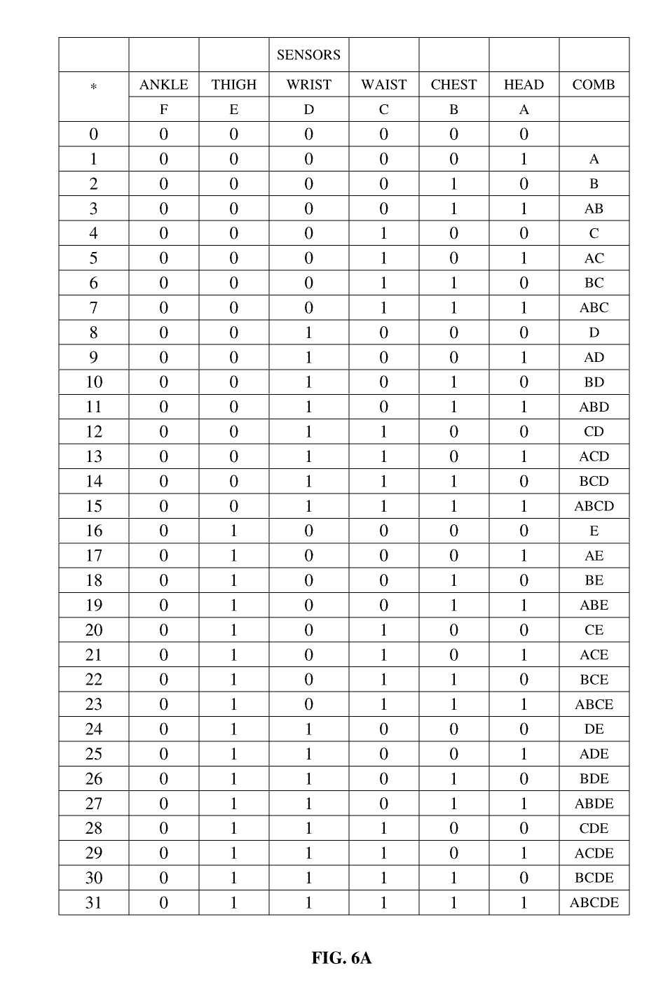

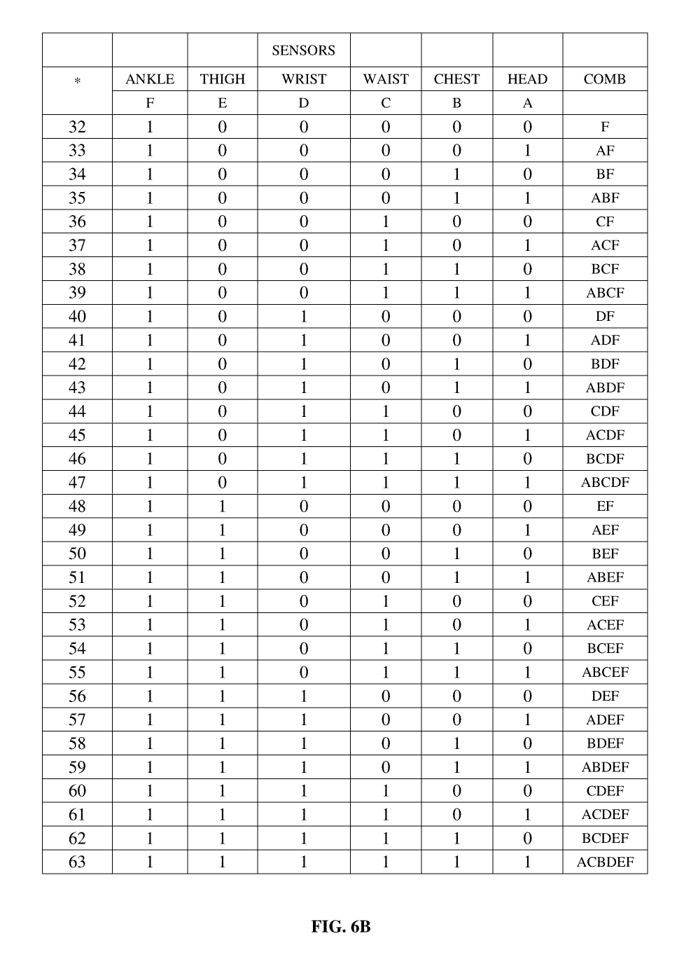

FIGS. 6A-6B exemplarily illustrate tables indicating positioning of a combination of multiple motion sensors 502 exemplarily illustrated in FIG. 5, on locations corresponding to different body parts of a body of a target object, for example, a patient undergoing stem cell treatment. The stem cell treatment monitoring system (SCTMS) 305 exemplarily illustrated in FIG. 3, assigns a label, for example, an alphabet to each individual motion sensor 502 within the wearable motion monitoring device 501 exemplarily illustrated in FIG. 5. For example, the SCTMS 305 labels the motion sensors 502 positioned on the head, the chest, the waist, the wrist, the thigh, and the ankle of the target object as A, B, C, D, E, and F respectively, as exemplarily illustrated in FIGS. 6A-6B. The tables in FIGS. 6A-6B also illustrate the combination of individual motion sensors 502 used for monitoring the physical progress of the target object undergoing the stem cell treatment. The combination of the alphabets and the numeric values 0 and 1 in the tables in FIGS. 6A-6B indicates the positions on the body parts and the combination of the motion sensors 502 active at a given time in the wearable motion monitoring device 501. For example, the value 000011 indicates that the motion sensors A and B positioned on the head and the chest of the body of the target object are active. The possible combinations of the motion sensors 502 on the body parts of the target object are evaluated with different machine learning algorithms, for example, the k-nearest neighbor (k-NN) classifier, the Bayesian decision making (BDM) algorithm, the support vector machine (SVM) algorithm, the least squares method (LSM), dynamic time warping (DTW), and artificial neural networks (ANNs). Each algorithm is applied to single, double, triple, quadruple, quintuple, and sextuple sensor configurations. These configurations create 63 different combinations of motion sensors 502, and for six machine learning techniques, a total of 63×6=378 combinations is examined. In an example, the sensor combination ABC is found to be the most suitable for monitoring movement of body parts, for example, the waist, spine, etc., to monitor physical progress of the target object undergoing stem cell treatment for a spinal cord injury.

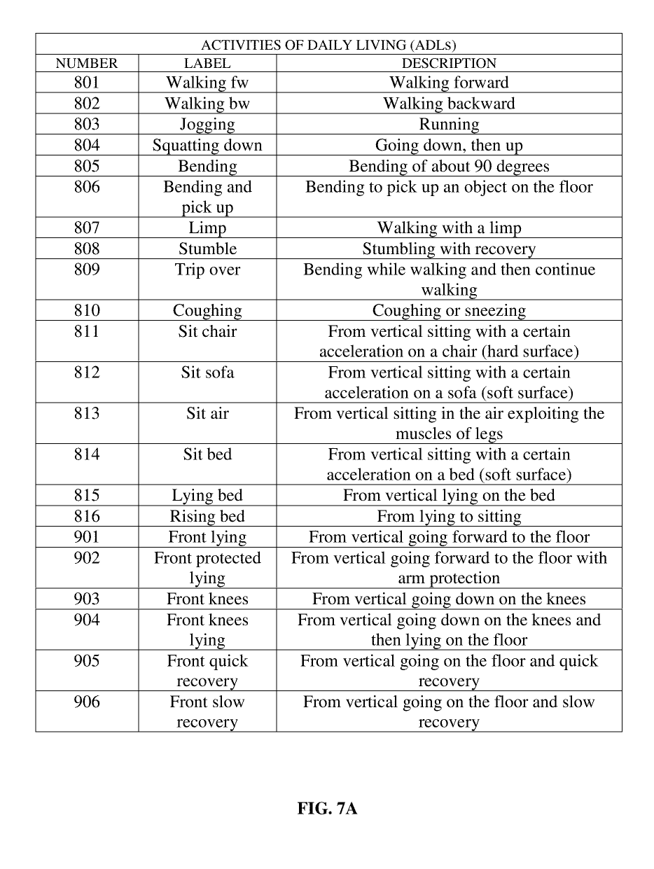

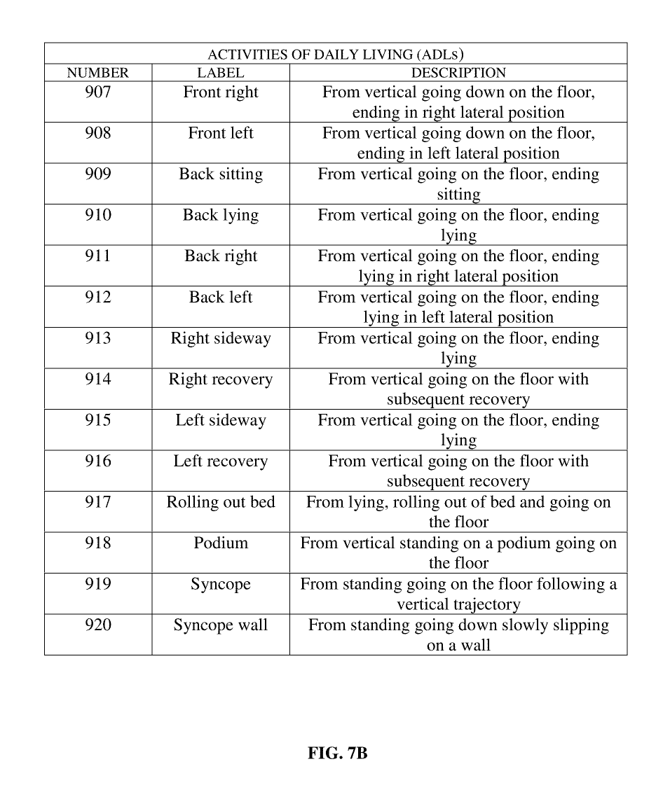

FIGS. 7A-7B exemplarily illustrate tables containing descriptions of movements related to activities of daily living of a target object undergoing stem cell treatment, to be monitored. The stem cell treatment monitoring system (SCTMS) 305 exemplarily illustrated in FIG. 3, classifies daily activities of the target object according to activities of daily living (ADLs) movement and assigns an identifier to physical movements of the target object undergoing the stem cell treatment based on movements of different body parts of the body of the target object to perform physical activities in a daily life of the target object. For example, the SCTMS 305 assigns the number 806 as an identifier for performing the activity of bending and picking up an object on the floor and the SCTMS 305 labels the activity as bending and pick up.

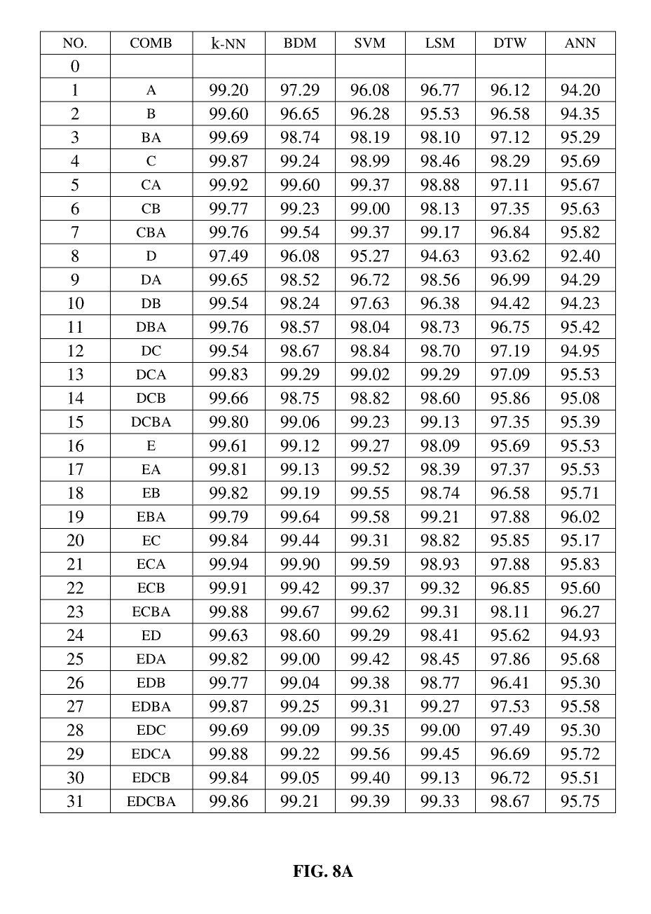

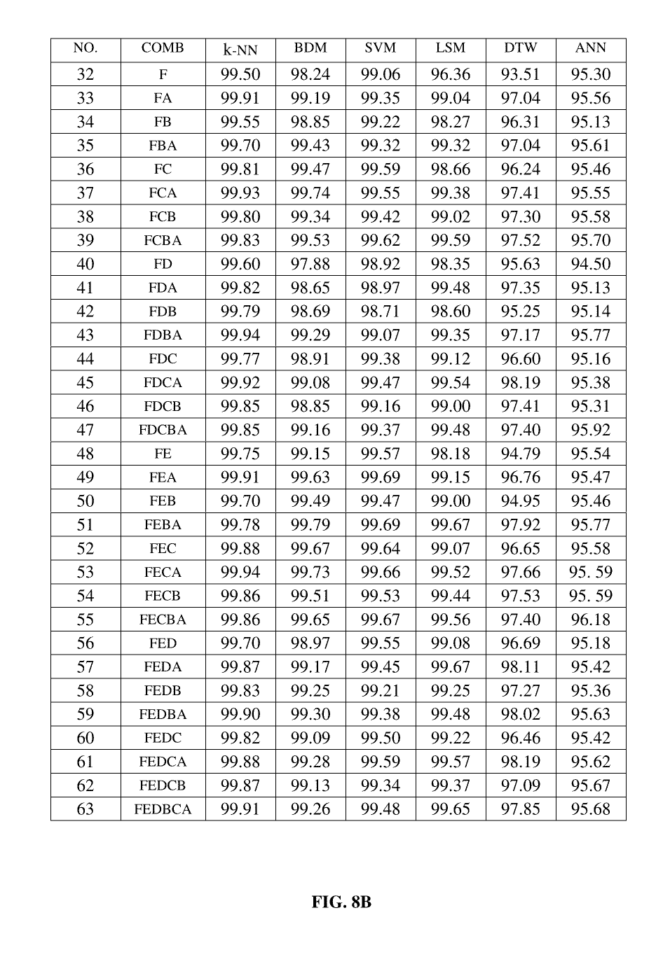

FIGS. 8A-8B exemplarily illustrate tables containing accuracy performances of multiple machine learning algorithms based on a combination of motion sensors 502 exemplarily illustrated in FIG. 5. The accuracy of the combination of the motion sensors 502 with their positions on the body parts of the target object is determined using multiple machine learning algorithms comprising, for example, the k-nearest neighbor classifier (k-NN), the Bayesian decision making (BDM) algorithm, a support vector machine (SVM), least squares method (LSM), dynamic time warping (DTW), an artificial neural network (ANN), etc. These algorithms determine the most suitable sensor configuration comprising single, double, triple, etc., and the position of the motion sensors 502 to monitor the physical progress of the target object. The stem cell treatment monitoring system (SCTMS) 305 exemplarily illustrated in FIG. 3, implements the k-nearest neighbor classifier (k-NN), support vector machines, etc., to check the accuracy of the measurement of the motor activity of the target object undergoing stem cell treatment using the combination of the motion sensors 502. For example, the SCTMS 305 determines the accuracy of motor activity measurement by a combination of the motion sensors 502 comprising BA and CA using the k-NN algorithm as 99.69 percent and 99.92 percent respectively. In an example, a patient undergoing stem cell treatment for a spinal cord injury is monitored using the SCTMS 305. The patient wears the wearable motion monitoring device 501 exemplarily illustrated in FIG. 5, and performs physical movement activities. The motion sensors 502 in the wearable motion monitoring device 501 detect the change in micro-movements of the patient. The SCTMS 305, using the machine learning algorithms, determines the configuration of the motion sensors A, B, and C positioned near the head, the chest, and the waist of the target object to monitor and measure a wrist movement of the target object suffering from the spinal cord injury.

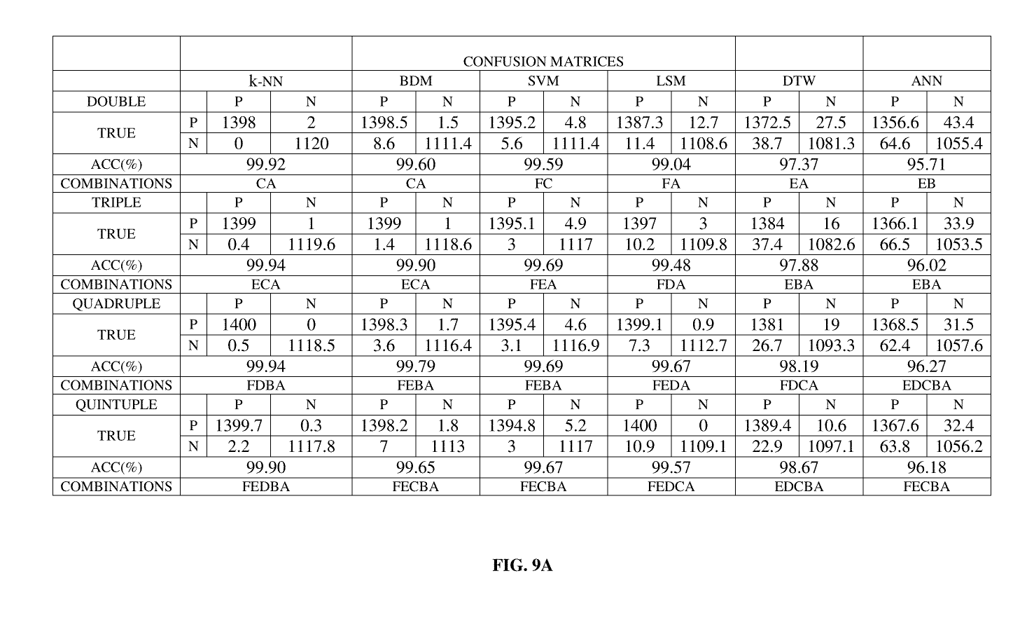

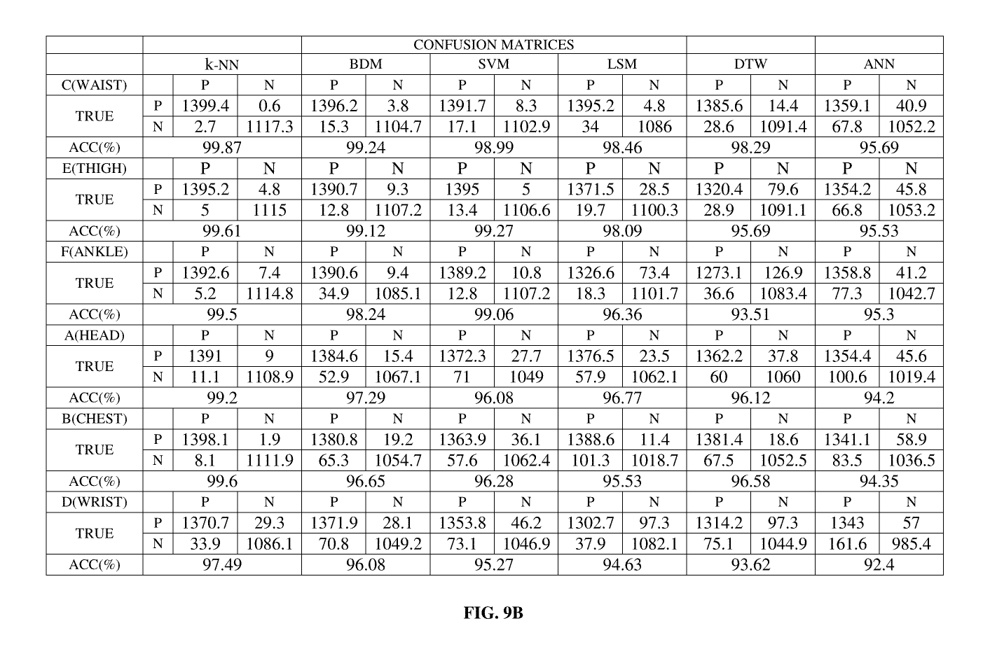

FIGS. 9A-9B exemplarily illustrate confusion matrix tables containing optimal results of double, triple, quadruple and quintuple motion sensor configurations under individual machine learning algorithms. The stem cell treatment monitoring system (SCTMS) 305 exemplarily illustrated in FIG. 3, uses multiple machine learning algorithms, for example, k-nearest neighbor classifier (k-NN), support vector machines, etc., to check the accuracy of the measurement of the motor activity of the target object undergoing stem cell treatment using the combination of motion sensors 502 exemplarily illustrated in FIG. 5. For example, the SCTMS 305 determines the accuracy of the motor activity data measured by the combination of motion sensors 502 such as ECA and FDBA using the k-NN algorithm as 99.94 percent and 99.94 percent respectively.

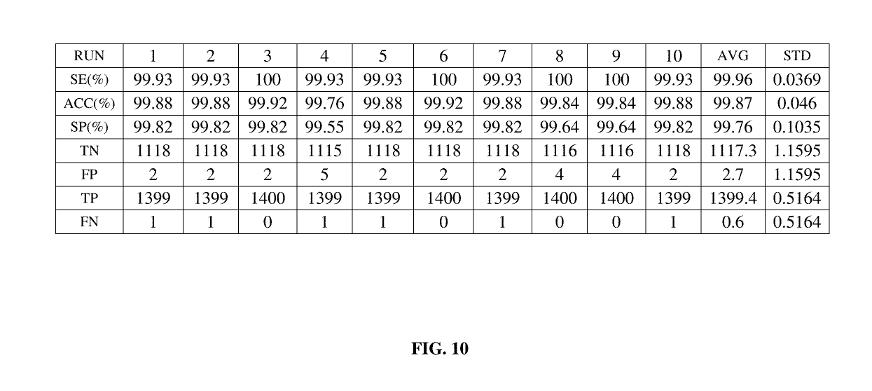

FIG. 10 exemplarily illustrates a table containing k-nearest neighbor classifier results of ten successive measurements made by a motion sensor 502 exemplarily illustrated in FIG. 5, positioned on a waist of a target object. The stem cell treatment monitoring system (SCTMS) 305 exemplarily illustrated in FIG. 3, positions the motion sensors 502 exemplarily illustrated in FIG. 5, on the waist of the target object undergoing stem cell treatment for a spinal cord injury to measure physical movement of the waist of the target for ten successive measurements. The SCTMS 305 performs ten successive measurements using the k-nearest neighbor classifier, calculates average and standard deviation of the ten successive measurements, and tabulates the readings as exemplarily illustrated in FIG. 10.

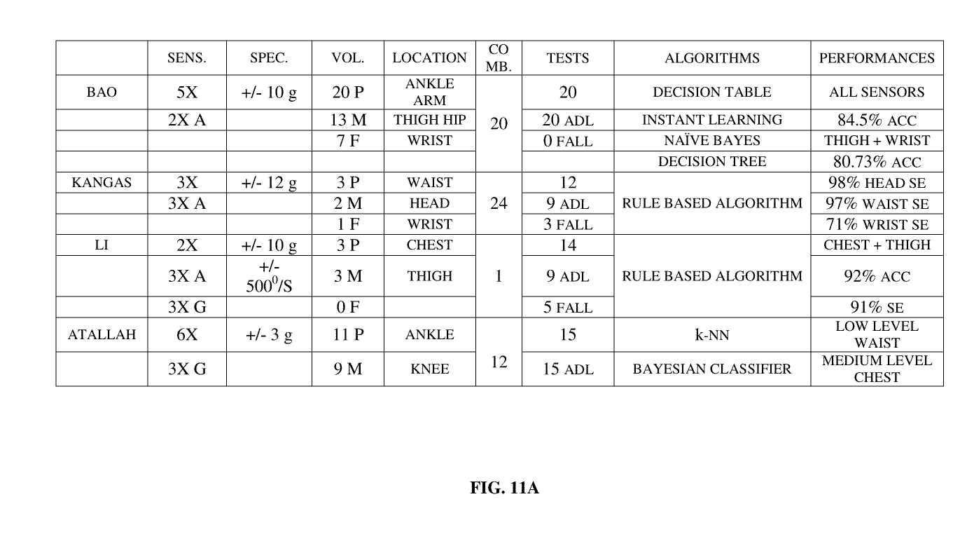

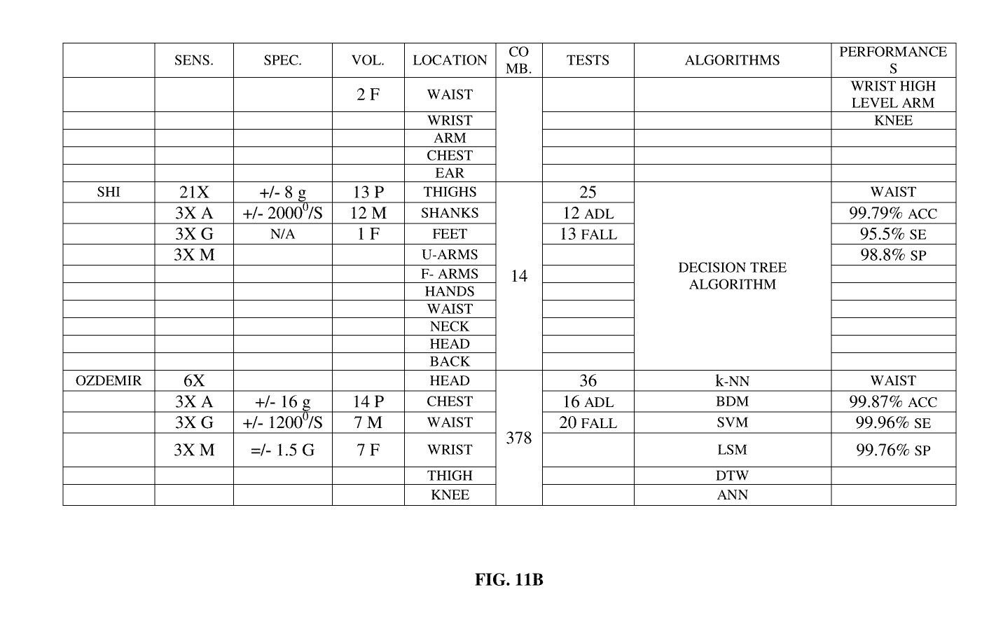

FIGS. 11A-11B exemplarily illustrate tables containing a summary of the motion sensors 502 exemplarily illustrated in FIG. 5, specifications of the motion sensors 502, combinations of the motion sensors 502, machine learning algorithms executed for measuring motor activity data of a target object undergoing stem cell treatment, and performances of the machine learning algorithms. The stem cell treatment monitoring system (SCTMS) 305 exemplarily illustrated in FIG. 3, uses different motion sensors 502 with different specifications to measure motor activity data of the target object. The SCTMS 305 uses different combinations of motion sensors 502 to measure motor activity data of different body parts of the target object. The SCTMS 305 processes the measured motor activity data using different machine learning algorithms and determines the accuracy of the measurement of motor activity using the combination of the motion sensors 502. For example, the SCTMS 305 uses the Bayesian decision making algorithm to measure and position motion sensors 5× with a +/−10 g specification on the ankle, arm, thigh, hip, and wrist of the target object undergoing stem cell treatment, records measurements 20, 20-activities of daily life, and 0-fall, and determines the accuracy of measurement of the motion sensors 502 as 84.5 percent.

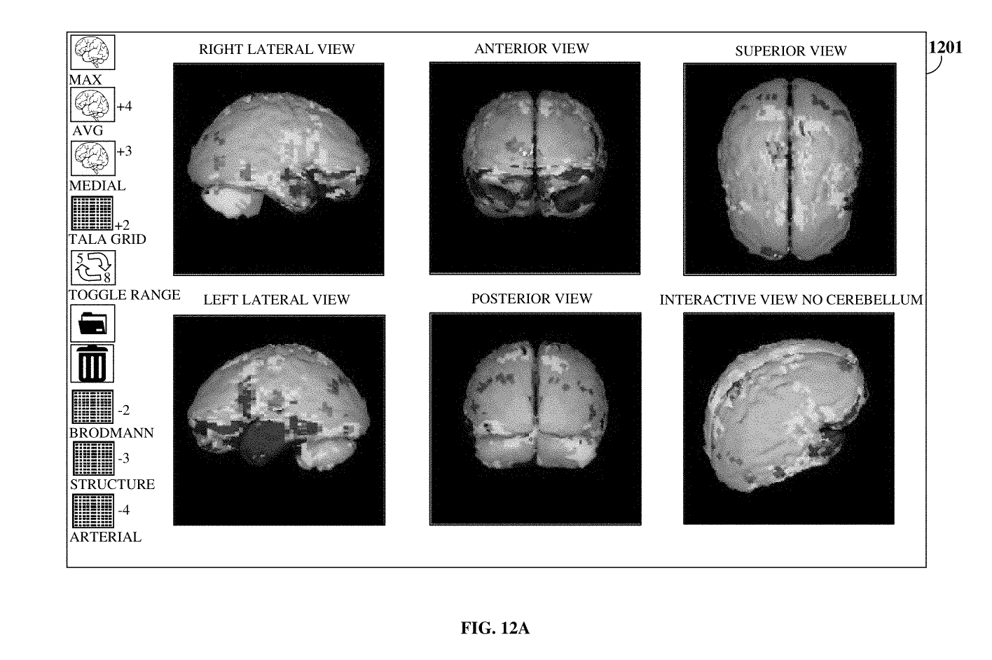

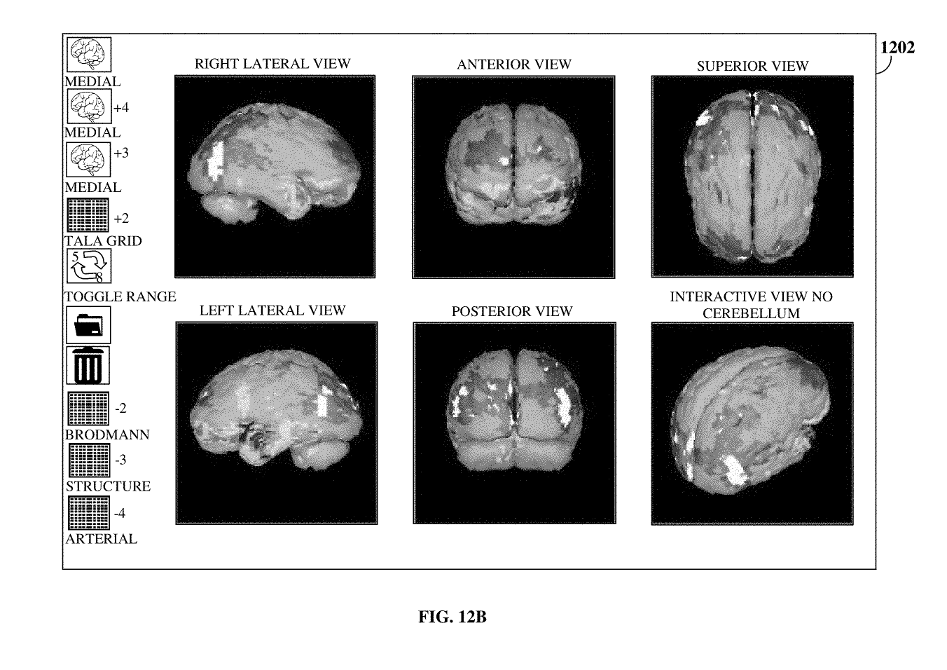

FIGS. 12A-12B exemplarily illustrate single-photon emission computed tomography (SPECT) images 1201 and 1202 of a brain undergoing stem cell treatment used by the stem cell treatment monitoring system (SCTMS) 305 exemplarily illustrated in FIG. 3, for monitoring physical progress of the target object. The SPECT images 1201 and 1202 show three-dimensional views, for example, a right lateral view, a left lateral view, an anterior view, a posterior view, a superior view, and an interactive view of the brain before and after a stem cell treatment, to measure localized changes in neural activity in the brain of the target object. The localized changes are represented by multiple colors in the SPECT images 1201 and 1202 as disclosed in detail description of FIG. 2.

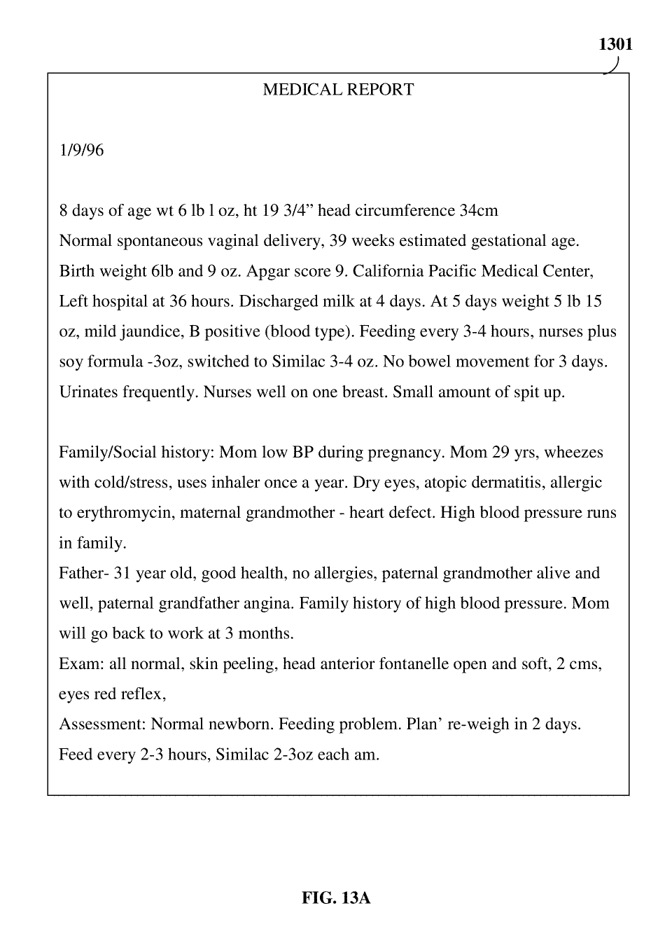

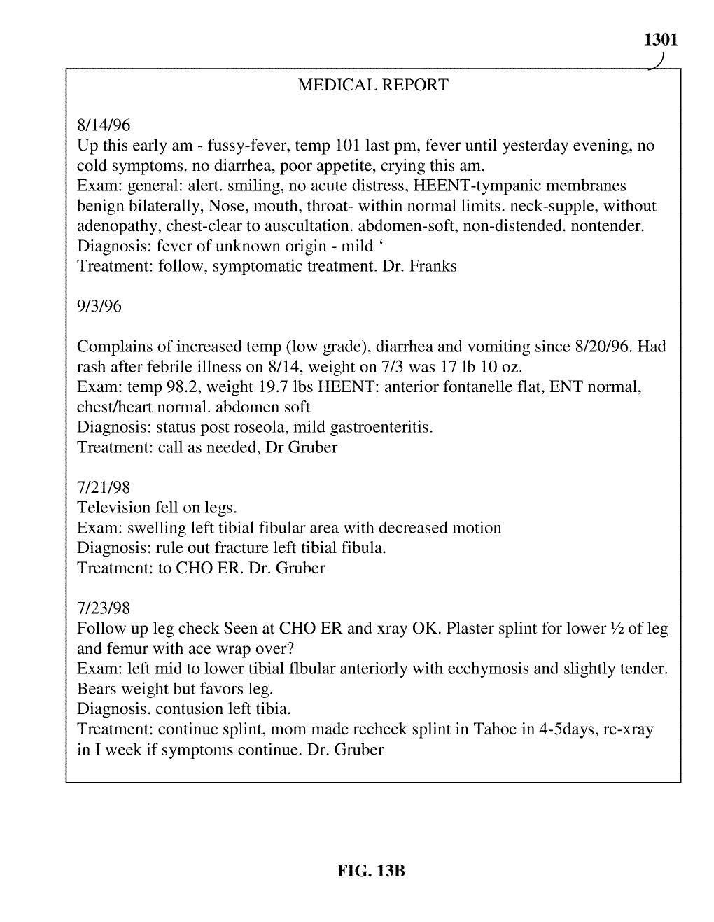

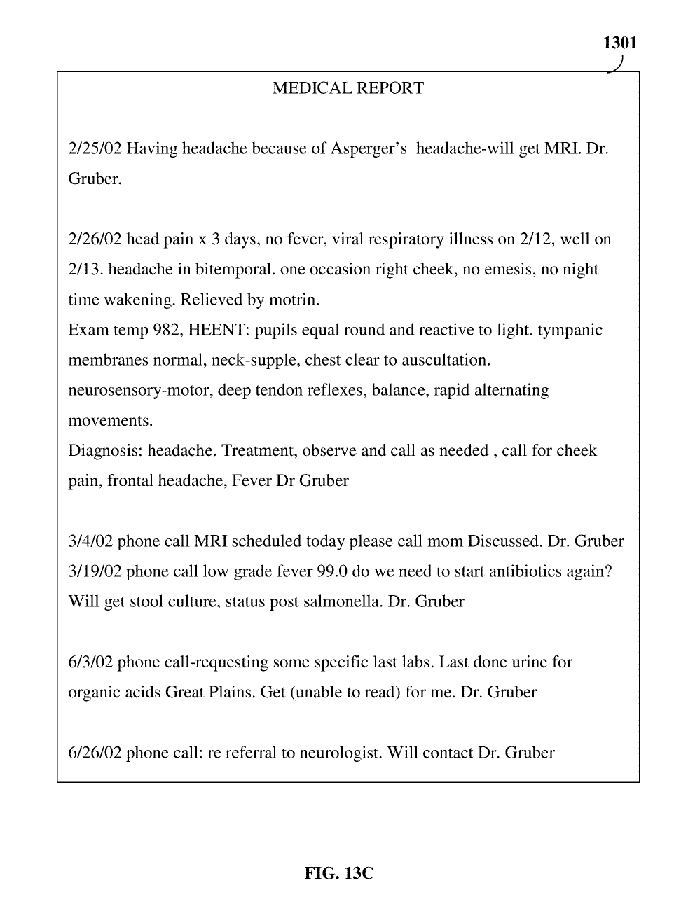

FIGS. 13A-13C exemplarily illustrate supplementary data of a target object used by the stem cell treatment monitoring system (SCTMS) 305 exemplarily illustrated in FIG. 3, for monitoring physical progress of the target object. The supplementary data, for example, a medical report 1301 provides a detailed medical history of the target object undergoing the stem cell treatment to allow a doctor to diagnose the medical condition of the patient. The SCTMS 305 analyzes the supplementary data, for example, a medical report 1301 for performing a baseline analysis and generating a physical progress report 306 exemplarily illustrated in FIG. 3, for the target object undergoing the stem cell treatment. For example, the doctor diagnoses a patient with a swelling in a left tibial fibular area as specified in the medical report 1301 of the patient, checks whether the patient has decreased motion by measuring the motor activity data through the motion sensors 502 in the wearable motion monitoring device 501 exemplarily illustrated in FIG. 5, and performs a baseline analysis to rule out fracture of the left tibial fibula.

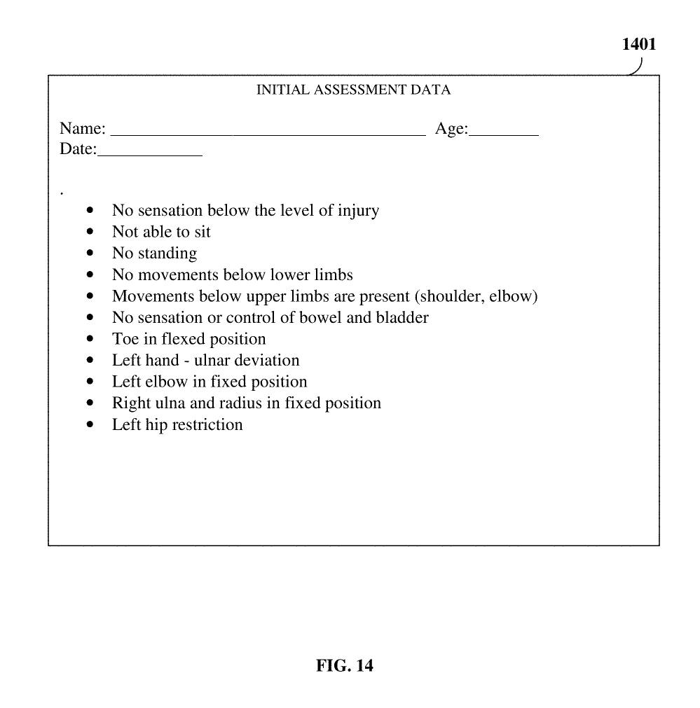

FIG. 14 exemplarily illustrates initial assessment data 1401 generated by the stem cell treatment monitoring system (SCTMS) 305 exemplarily illustrated in FIG. 3, during performance of a baseline analysis of physical progress of a target object undergoing the stem cell treatment. The SCTMS 305 records the physiological condition parameters of the target object undergoing the stem cell treatment for a medical condition on the predetermined stem cell treatment checklist rendered on the graphical user interface (GUI) displayed on the electronic device 304 exemplarily illustrated in FIG. 3. The SCTMS 305 analyzes the recorded physiological condition parameters of the target object and performs a baseline analysis of the medical condition of the target object. Consider an example where a patient with a spinal cord injury (SCI) undergoes stem cell treatment. The doctor records the physiological condition parameters comprising, for example, lack of response from muscles, involuntary, unsustained muscle movement, muscle movement in gravity eliminated condition, etc., of the patient using the SCI stem cell treatment injury checklist 301b exemplarily illustrated in FIG. 4B. The SCTMS 305 analyzes the dynamically received physiological condition parameters and generates a baseline analysis comprising, for example, no sensation below the level of injury, not able to sit, no standing, etc., of the patient undergoing the stem cell treatment as disclosed in the detail description of FIG. 2.

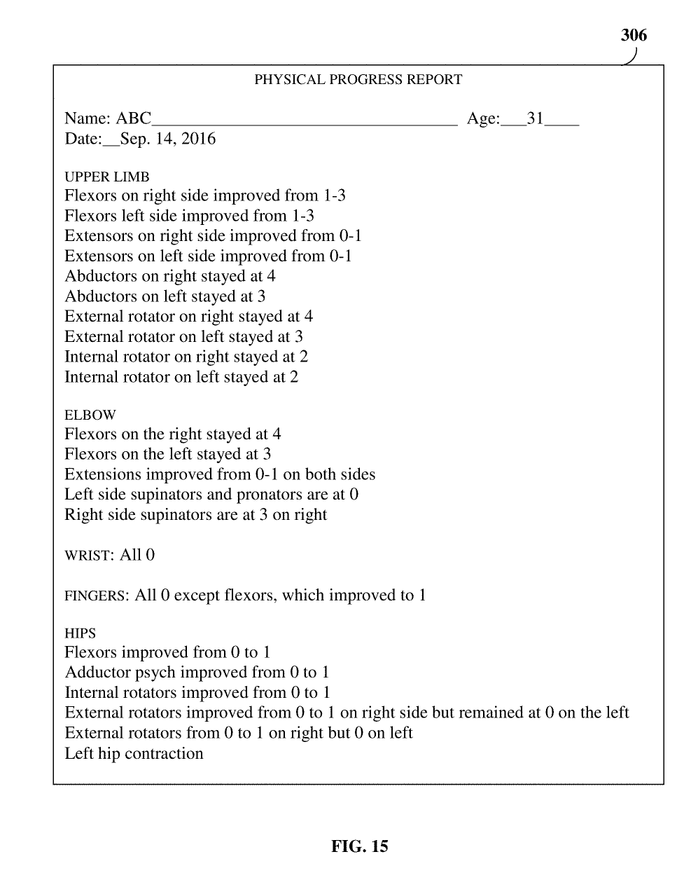

FIG. 15 exemplarily illustrates a physical progress report 306 generated by the stem cell treatment monitoring system (SCTMS) 305 exemplarily illustrated in FIG. 3, based on the baseline analysis of physical progress of a target object undergoing the stem cell treatment. The SCTMS 305 generates the physical progress report 306 of the target object based on the initial assessment data 1401 exemplarily illustrated in FIG. 14, generated based on the baseline analysis of the target object performed by the SCTMS 305. For example, the physical progress report 306 of a patient undergoing stem cell treatment comprises flexors on right side of upper limb improved from 1-3, flexors left side of upper limb improved from 1-3, extensors on right side of upper limb improved from 0-1, extensors on left side of upper limb improved from 0-1, etc., as disclosed in detailed description of FIG. 2.

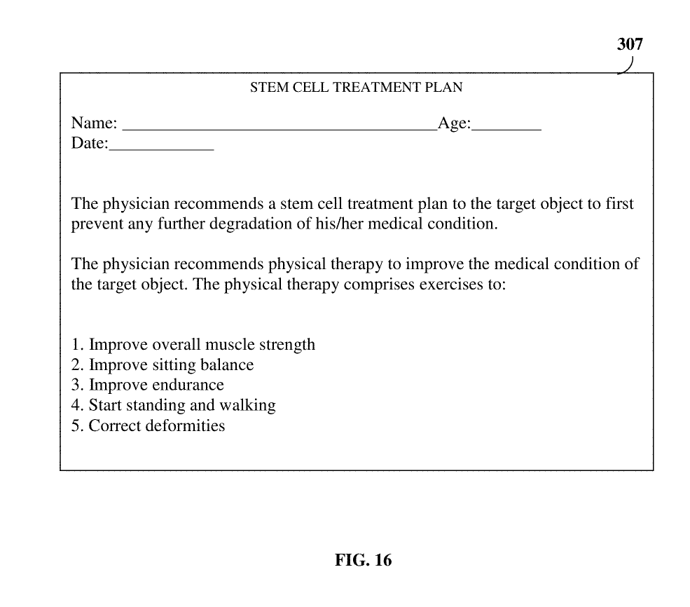

FIG. 16 exemplarily illustrates a stem cell treatment plan 307 generated by the stem cell treatment monitoring system (SCTMS) 305 based on a physical progress report 306 exemplarily illustrated in FIG. 3, of a target object undergoing the stem cell treatment. The SCTMS 305 generates the stem cell treatment plan 307 of the target by analyzing the generated physical progress report 306 of the target object. For example, the SCTMS 305 generates a stem cell treatment plan 307 for a patient undergoing stem cell therapy for a spinal cord injury, recommending physical therapy to improve overall muscle strength, improve sitting balance, improve endurance, start standing and walking, etc., as disclosed in detailed description of FIG. 2.

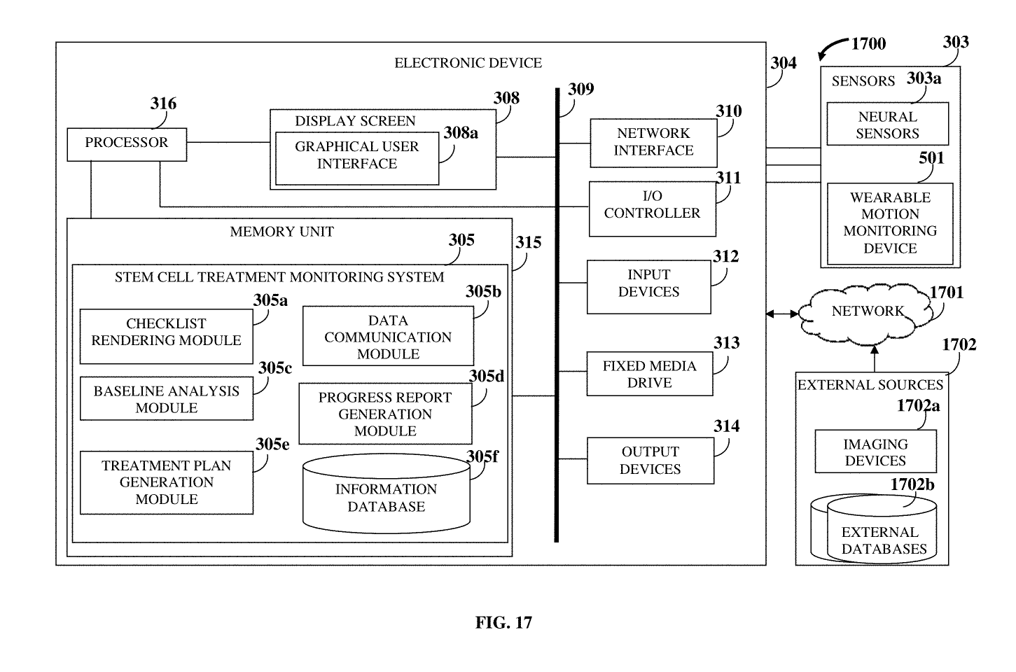

FIG. 17 exemplarily illustrates a system 1700 comprising the sensors 303 and the stem cell treatment monitoring system (SCTMS) 305 implemented on an electronic device 304 for monitoring physical progress of a target object undergoing stem cell treatment for determining effectiveness of the stem cell treatment and adapting the stem cell treatment. In the system 1700 disclosed herein, the SCTMS 305 on the electronic device 304 interfaces with the sensors 303, that is, the neural sensors 303a and the wearable motion monitoring device 501 with the motion sensors 502 exemplarily illustrated in FIG. 5, worn by the target object, for monitoring physical progress of a target object undergoing stem cell treatment for determining effectiveness of the stem cell treatment and adapting the stem cell treatment, and therefore more than one hardware device other than a generic computer is used in the system 1700. The sensors 303 are in operable communication with the SCTMS 305 on the electronic device 304.

The electronic device 304 is configured as a user device, for example, a personal computer, a tablet computing device, a mobile computer, a mobile phone, a smartphone, a portable computing device, a personal digital assistant, a laptop, a wearable computing device such as the Google Glass® of Google Inc., the Apple Watch® of Apple Inc., etc., a touch centric device, a client device, a portable electronic device, a network enabled computing device, an interactive network enabled communication device, an image capture device, any other suitable computing equipment, combinations of multiple pieces of computing equipment, etc. In an embodiment, the electronic device 304 is a hybrid computing device that combines the functionality of multiple devices. Examples of a hybrid computing device comprise a cellular telephone that includes a media player functionality, a cellular telephone that includes multimedia functions, and a portable device that receives electronic mail (email), supports mobile telephone calls, has a media player functionality, and supports web browsing. In an embodiment, computing equipment is used to implement applications such as media playback applications, a web browser, an electronic mail (email) application, a calendar application, etc. The electronic device 304 deploys the stem cell treatment monitoring system (SCTMS) 305 for monitoring the physical progress of a target object undergoing stem cell treatment for determining the effectiveness of the stem cell treatment and adapting the stem cell treatment.

The electronic device 304 is a computer system that is programmable using a high level computer programming language. In an embodiment, the stem cell treatment monitoring system (SCTMS) 305 is implemented on the electronic device 304 using programmed and purposeful hardware. The electronic device 304 communicates with one or more external sources 1702, for example, imaging devices 1702a such as scanners, external databases 1702b, etc., via a network 1701, for example, a short range network or a long range network. The network 1701 is, for example, the internet, an intranet, a wired network, a wireless network, a network that implements Bluetooth® of Bluetooth Sig, Inc., a network that implements Wi-Fi® of Wi-Fi Alliance Corporation, an ultra-wideband communication network (UWB), a wireless universal serial bus (USB) communication network, a communication network that implements ZigBee® of ZigBee Alliance Corporation, a general packet radio service (GPRS) network, a mobile telecommunication network such as a global system for mobile (GSM) communications network, a code division multiple access (CDMA) network, a third generation (3G) mobile communication network, a fourth generation (4G) mobile communication network, a fifth generation (5G) mobile communication network, a long-term evolution (LTE) mobile communication network, a public telephone network, etc., a local area network, a wide area network, an internet connection network, an infrared communication network, etc., or a network formed from any combination of these networks. In an embodiment, the SCTMS 305 is accessible to a user, for example, a medical practitioner through a broad spectrum of technologies and devices, for example, cellular phones, tablet computing devices, etc., with access to the internet.

As exemplarily illustrated in FIG. 17, the electronic device 304 comprises a non-transitory computer readable storage medium, for example, a memory unit 315 for storing program instructions, applications, and data, and at least one processor 316 communicatively coupled to the non-transitory computer readable storage medium. As used herein, the “non-transitory computer readable storage medium” refers to all computer readable media, for example, non-volatile media, volatile media, and transmission media, except for a transitory, propagating signal. Non-volatile media comprise, for example, solid state drives, optical discs or magnetic disks, and other persistent memory volatile media including a dynamic random access memory (DRAM), which typically constitute a main memory. Volatile media comprise, for example, a register memory, a processor cache, a random access memory (RAM), etc. Transmission media comprise, for example, coaxial cables, copper wire, fiber optic cables, modems, etc., including wires that constitute a system bus coupled to the processor 316. The non-transitory computer readable storage medium is configured to store computer program instructions defined by modules, for example, 305a, 305b, 305c, 305d, 305e, etc., of the stem cell treatment monitoring system (SCTMS) 305. In an embodiment as exemplarily illustrated in FIG. 17, the SCTMS 305 is installed and stored in the memory unit 315 of the electronic device 304. The memory unit 315 is, for example, a random access memory (RAM) or another type of dynamic storage device that stores information and instructions for execution by the processor 316. The memory unit 315 also stores temporary variables and other intermediate information used during execution of the instructions by the processor 316. The electronic device 304 further comprises a read only memory (ROM) or another type of static storage device that stores static information and instructions for the processor 316.

The processor 316 is configured to execute the computer program instructions defined by the modules, for example, 305a, 305b, 305c, 305d, 305e, etc., of the stem cell treatment monitoring system (SCTMS) 305. The processor 316 refers to any one or more microprocessors, central processing unit (CPU) devices, finite state machines, computers, microcontrollers, digital signal processors, logic, a logic device, an user circuit, an application specific integrated circuit (ASIC), a field-programmable gate array (FPGA), a chip, etc., or any combination thereof, capable of executing computer programs or a series of commands, instructions, or state transitions. In an embodiment, the processor 316 is implemented as a processor set comprising, for example, a programmed microprocessor and a math or graphics co-processor. The processor 316 is selected, for example, from the Intel® processors such as the Itanium® microprocessor or the Pentium® processors, Advanced Micro Devices (AMD®) processors such as the Athlon® processor, UltraSPARC® processors, microSPARC® processors, hp® processors, International Business Machines (IBM®) processors such as the PowerPC® microprocessor, the MIPS® reduced instruction set computer (RISC) processor of MIPS Technologies, Inc., RISC based computer processors of ARM Holdings, Motorola® processors, Qualcomm® processors, etc. The SCTMS 305 disclosed herein is not limited to employing a processor 316. In an embodiment, the SCTMS employs a controller or a microcontroller. The processor 316 executes the modules, for example, 305a, 305b, 305c, 305d, 305e, etc., of the SCTMS 305.

As exemplarily illustrated in FIG. 17, the electronic device 304 further comprises a display screen 308 that displays the graphical user interface (GUI) 308a provided by the stem cell treatment monitoring system (SCTMS) 305. The display screen 308 comprises, for example, a video display, a liquid crystal display, a plasma display, an organic light emitting diode (OLED) based display, etc. The GUI 308a is, for example, an online web interface, a web based downloadable application interface, a mobile based downloadable application interface, etc. The GUI 308a displays the results of computations performed by the modules 305a, 305b, 305c, 305d, 305e, etc., of the SCTMS 305. The display screen 308, via the GUI 308a, displays information, display interfaces, user interface elements such as swipable arrows, icons, checkboxes, etc., for example, for inputting checks on the predefined condition based stem cell treatment checklist 301 exemplarily illustrated in FIG. 3, displaying the physical progress report 306 generated by the SCTMS 305 exemplarily illustrated in FIG. 3, displaying the stem cell treatment plan 307 generated by the SCTMS 305 exemplarily illustrated in FIG. 3, etc. The SCTMS 305 renders the GUI 308a on the display screen 308 to display the physical progress report 306, the stem cell treatment plan 307, etc., of the target object undergoing the stem cell treatment.

The electronic device 304 further comprises a data bus 309, a network interface 310, an input/output (I/O) controller 311, input devices 312, a fixed media drive 313 such as a hard drive, output devices 314, etc. The data bus 309 permits communications between the modules, for example, 308, 310, 311, 312, 313, 314, 315, 316, etc., of the electronic device 304. The network interface 310 enables connection of the stem cell treatment monitoring system (SCTMS) 305 in the electronic device 304 to the network 1701 and to the sensors 303. In an embodiment, the network interface 310 is provided as an interface card also referred to as a line card. The network interface 310 comprises, for example, one or more of an infrared (IR) interface, an interface implementing Wi-Fi® of Wi-Fi Alliance Corporation, a universal serial bus (USB) interface, a FireWire® interface of Apple Inc., an Ethernet interface, a frame relay interface, a cable interface, a digital subscriber line (DSL) interface, a token ring interface, a peripheral controller interconnect (PCI) interface, a local area network (LAN) interface, a wide area network (WAN) interface, interfaces using serial protocols, interfaces using parallel protocols, Ethernet communication interfaces, asynchronous transfer mode (ATM) interfaces, a high speed serial interface (HSSI), a fiber distributed data interface (FDDI), interfaces based on transmission control protocol (TCP)/internet protocol (IP), interfaces based on wireless communications technology such as satellite technology, radio frequency (RF) technology, near field communication, etc. The I/O controller 311 controls input actions and output actions performed by the SCTMS 305. The input devices 312 are used for inputting data into the SCTMS 305. The user uses the input devices 312 to provide inputs to the SCTMS 305. For example, the user configures the neural sensors 303a and the wearable motion monitoring device 501 positioned and/or worn by the target object undergoing the stem cell treatment via the SCTMS 305 using the input devices 312. In another example, the user records the physiological condition parameters of the target object in the predefined condition based stem cell treatment checklist 301 displayed on the graphical user interface (GUI) 308a using the input devices 312. The input devices 312 are, for example, a keyboard such as an alphanumeric keyboard, a microphone, a joystick, a pointing device such as a computer mouse, a touch pad, a light pen, a physical button, a touch sensitive display device, a track ball, a pointing stick, any device capable of sensing a tactile input, etc.

The stem cell treatment monitoring system (SCTMS) 305 is activated on the electronic device 304 via the graphical user interface (GUI) 308a. The SCTMS 305 comprises a checklist rendering module 305a, a data communication module 305b, a baseline analysis module 305c, a progress report generation module 305d, and a treatment plan generation module 305e stored in the memory unit 315 of the electronic device 304. The checklist rendering module 305a renders a predefined condition based stem cell treatment checklist 301 on the GUI 308a displayed on the electronic device 304. The data communication module 305b dynamically receives the neural activity data and the motor activity data of the target object recorded and transmitted by the sensors 303 to the electronic device 304 as disclosed in the detailed description of FIG. 2. The data communication module 305b also records physiological condition parameters of the target object in the rendered predefined condition based stem cell treatment checklist 301 using user inputs and the dynamically received neural activity data and the dynamically received motor activity data. In an embodiment, the data communication module 305b also receives one or more color coded tomographic images and supplementary data of the target object from the external sources 1702, for example, imaging devices 1702a, external databases 1702b, etc.

The baseline analysis module 305c performs a baseline analysis of the physical progress of the target object undergoing the stem cell treatment based on the recorded physiological condition parameters in real time. In an embodiment, the baseline analysis module 305c performs the baseline analysis of the physical progress of the target object undergoing the stem cell treatment by correlating the received color coded tomographic images and the received supplementary data with the recorded physiological condition parameters as disclosed in the detailed description of FIG. 2. The progress report generation module 305d generates a physical progress report 306 based on the baseline analysis of the physical progress of the target object for determining the effectiveness of the stem cell treatment and renders the generated physical progress report 306 on the graphical user interface (GUI) 308a of the electronic device 304 as disclosed in the detailed description of FIG. 2. The progress report generation module 305d stores the generated physical progress report 306 in an information database 305f. The treatment plan generation module 305e generates and renders a stem cell treatment plan 307 based on the rendered physical progress report 306 of the target object on the GUI 308a displayed on the electronic device 304. The treatment plan generation module 305e stores the generated stem cell treatment plan 307 in the information database 305f.

The stem cell treatment monitoring system (SCTMS) 305 stores the generated physical progress report 306, the generated stem cell treatment plan 307, and the supplementary data received from the external sources 1702, for example, the medical reports, the physical therapy reports, etc., of the target object undergoing the stem cell treatment in the information database 305f. The information database 305f of the SCTMS 305 can be any storage area or medium that can be used for storing data and files. In an embodiment, the SCTMS 305 stores logs in external databases 1702b, for example, a structured query language (SQL) data store or a not only SQL (NoSQL) data store such as the Microsoft® SQL Server®, the Oracle® servers, the MySQL® database of MySQL AB Company, the mongoDB® of MongoDB, Inc., the Neo4j graph database of Neo Technology Corporation, the Cassandra database of the Apache Software Foundation, the HBase™ database of the Apache Software Foundation, etc. In an embodiment, the information database 305f can also be a location on a file system. In another embodiment, the information database 305f can be remotely accessed by the SCTMS 305 via the network 1701. In another embodiment, the information database 305f is configured as a cloud based database implemented in a cloud computing environment, where computing resources are delivered as a service over the network 1701.

Computer applications and programs are used for operating the stem cell treatment monitoring system (SCTMS) 305. The programs are loaded onto the fixed media drive 313 and into the memory unit 315 of the electronic device 304. In an embodiment, the computer applications and programs are loaded directly on the electronic device 304 via the network 1701. Computer applications and programs are executed by double clicking a related icon displayed on the display screen 308 using one of the input devices 312. The output devices 314 output the results of operations performed by the SCTMS 305. For example, the SCTMS 305 renders the physical progress report 306 and the stem cell treatment plan 307 of the target object undergoing the stem cell treatment to a user of the SCTMS 305 using the output devices 314.

The processor 316 executes an operating system, for example, the Linux® operating system, the Unix® operating system, any version of the Microsoft® Windows® operating system, the Mac OS of Apple Inc., the IBM® OS/2, VxWorks® of Wind River Systems, Inc., QNX Neutrino® developed by QNX Software Systems Ltd., the Palm OS®, the Solaris operating system developed by Sun Microsystems, Inc., the Android® operating system of Google Inc., the Windows Phone® operating system of Microsoft Corporation, the BlackBerry® operating system of BlackBerry Limited, the iOS operating system of Apple Inc., the Symbian™ operating system of Symbian Foundation Limited, etc. The stem cell treatment monitoring system (SCTMS) 305 employs the operating system for performing multiple tasks. The operating system is responsible for management and coordination of activities and sharing of resources of the SCTMS 305. The operating system further manages security of the SCTMS 305, peripheral devices connected to the SCTMS 305, and network connections. The operating system employed on the SCTMS 305 recognizes, for example, inputs provided by the user of the electronic device 304 using one of the input devices 312, the output devices 314, files, and directories stored locally on the fixed media drive 313. The operating system on the SCTMS 305 executes different programs using the processor 316. The processor 316 and the operating system together define a computer platform for which application programs in high level programming languages are written. The operating system of the electronic device 304 determines the programming languages used in the SCTMS 305. For example, the Java® programming language is used for developing the SCTMS 305 on the electronic device 304 with an Android® operating system, while Objective-C® of Apple Inc., is used for developing the SCTMS 305 on the electronic device 304 with the iOS operating system, and the UNITY® libraries and platforms of Unity IPR ApS, LLC., are used for developing the SCTMS 305 for both the Android® operating system and the iOS operating system.

The processor 316 retrieves instructions defined by the checklist rendering module 305a, the data communication module 305b, the baseline analysis module 305c, the progress report generation module 305d, and the treatment plan generation module 305e for performing respective functions disclosed above. The processor 316 retrieves instructions for executing the modules, for example, 305a, 305b, 305c, 305d, 305e, etc., of the stem cell treatment monitoring system (SCTMS) 305 from the memory unit 315. A program counter determines the location of the instructions in the memory unit 315. The program counter stores a number that identifies the current position in the program of each of the modules, for example, 305a, 305b, 305c, 305d, 305e, etc., of the SCTMS 305. The instructions fetched by the processor 316 from the memory unit 315 after being processed are decoded. The instructions are stored in an instruction register in the processor 316. After processing and decoding, the processor 316 executes the instructions, thereby performing one or more processes defined by those instructions.

At the time of execution, the instructions stored in the instruction register are examined to determine the operations to be performed. The processor 316 then performs the specified operations. The operations comprise arithmetic operations and logic operations. The operating system performs multiple routines for performing a number of tasks required to assign the input devices 312, the output devices 314, and the memory unit 315 for execution of the modules, for example, 305a, 305b, 305c, 305d, 305e, etc., of the stem cell treatment monitoring system (SCTMS) 305. The tasks performed by the operating system comprise, for example, assigning memory to the modules, for example, 305a, 305b, 305c, 305d, 305e, etc., of the SCTMS 305, and to data used by the SCTMS 305, moving data between the memory unit 315 and disk units, and handling input/output operations. The operating system performs the tasks on request by the operations and after performing the tasks, the operating system transfers the execution control back to the processor 316. The processor 316 continues the execution to obtain one or more outputs. The outputs of the execution of the modules, for example, 305a, 305b, 305c, 305d, 305e, etc., of the SCTMS 305 are displayed to the user on the output devices 314.

The non-transitory computer readable storage medium disclosed herein stores computer program codes comprising instructions executable by at least one processor 316 for monitoring physical progress of a target object undergoing stem cell treatment for determining effectiveness of the stem cell treatment and adapting the stem cell treatment. The computer program codes comprise a first computer program code for rendering a predefined condition based stem cell treatment checklist 301 on the graphical user interface (GUI) 308a displayed on the display screen 308 of the electronic device 304; a second computer program code for dynamically receiving neural activity data and motor activity data of the target object recorded by multiple sensors 303 in operable communication with the electronic device 304; a third computer program code for recording physiological condition parameters of the target object in the rendered predefined condition based stem cell treatment checklist 301 using user inputs and the dynamically received neural activity data and the dynamically received motor activity data; a fourth computer program code for performing a baseline analysis of the physical progress of the target object undergoing the stem cell treatment based on the recorded physiological condition parameters in real time; a fifth computer program code for generating a physical progress report 306 based on the baseline analysis of the physical progress of the target object for determining the effectiveness of the stem cell treatment, and rendering the generated physical progress report 306 on the GUI 308a displayed on the display screen 308 of the electronic device 304; and a sixth computer program code for generating and rendering a stem cell treatment plan 307 based on the generated physical progress report 306 of the target object on the GUI 308a displayed on the display screen 308 of the electronic device 304.

In an embodiment, the computer program codes further comprise a seventh computer program code for receiving one or more color coded tomographic images and supplementary data of the target object from one or more external sources 1702. In this embodiment, the fourth computer program code further comprises an eighth computer program code for correlating the received color coded tomographic images and the received supplementary data with the recorded physiological condition parameters to perform the baseline analysis of the physical progress of the target object undergoing the stem cell treatment.

The computer program codes further comprise one or more additional computer program codes for performing additional steps that may be required and contemplated for monitoring physical progress of a target object undergoing stem cell treatment for determining effectiveness of the stem cell treatment and adapting the stem cell treatment. In an embodiment, a single piece of computer program code comprising computer executable instructions performs one or more steps of the method disclosed herein for monitoring physical progress of a target object undergoing stem cell treatment for determining effectiveness of the stem cell treatment and adapting the stem cell treatment. The computer program codes comprising computer executable instructions are embodied on the non-transitory computer readable storage medium. The processor 316 of the electronic device 304 retrieves these computer executable instructions and executes them. When the computer executable instructions are executed by the processor 316, the computer executable instructions cause the processor 316 to perform the steps of the method for monitoring physical progress of a target object undergoing stem cell treatment for determining effectiveness of the stem cell treatment and adapting the stem cell treatment.

It will be readily apparent in different embodiments that the various methods, algorithms, and computer programs disclosed herein are implemented on non-transitory computer readable storage media appropriately programmed for computing devices. The non-transitory computer readable storage media participate in providing data, for example, instructions that are read by a computer, a processor or a similar device. In different embodiments, the “non-transitory computer readable storage media” also refer to a single medium or multiple media, for example, a centralized database, a distributed database, and/or associated caches and servers that store one or more sets of instructions that are read by a computer, a processor or a similar device. The “non-transitory computer readable storage media” also refer to any medium capable of storing or encoding a set of instructions for execution by a computer, a processor or a similar device and that causes a computer, a processor or a similar device to perform any one or more of the methods disclosed herein. Common forms of the non-transitory computer readable storage media comprise, for example, a floppy disk, a flexible disk, a hard disk, magnetic tape, a laser disc, a Blu-ray Disc® of the Blu-ray Disc Association, any magnetic medium, a compact disc-read only memory (CD-ROM), a digital versatile disc (DVD), any optical medium, a flash memory card, punch cards, paper tape, any other physical medium with patterns of holes, a random access memory (RAM), a programmable read only memory (PROM), an erasable programmable read only memory (EPROM), an electrically erasable programmable read only memory (EEPROM), a flash memory, any other memory chip or cartridge, or any other medium from which a computer can read.

In an embodiment, the computer programs that implement the methods and algorithms disclosed herein are stored and transmitted using a variety of media, for example, the computer readable media in a number of manners. In an embodiment, hard-wired circuitry or custom hardware is used in place of, or in combination with, software instructions for implementing the processes of various embodiments. Therefore, the embodiments are not limited to any specific combination of hardware and software. The computer program codes comprising computer executable instructions can be implemented in any programming language. Examples of programming languages that can be used comprise C, C++, C#, Java®, Perl®, Python®, Microsoft® .NET, Objective-C®, etc. Other object-oriented, functional, scripting, and/or logical programming languages can also be used. In an embodiment, the computer program codes or software programs are stored on or in one or more mediums as object code. In another embodiment, various aspects of the method and the system 1700 disclosed herein are implemented in a non-programmed environment comprising documents created, for example, in a hypertext markup language (HTML), an extensible markup language (XML), or other format that render aspects of the graphical user interface (GUI) 308a or perform other functions, when viewed in a visual area or a window of a browser program. In another embodiment, various aspects of the method and the system 1700 disclosed herein are implemented as programmed elements, or non-programmed elements, or any suitable combination thereof.

Where databases are described such as the information database 305f, it will be understood by one of ordinary skill in the art that (i) alternative database structures to those described may be employed, and (ii) other memory structures besides databases may be employed. Any illustrations or descriptions of any sample databases disclosed herein are illustrative arrangements for stored representations of information. In an embodiment, any number of other arrangements are employed besides those suggested by tables illustrated in the drawings or elsewhere. Similarly, any illustrated entries of the databases represent exemplary information only; one of ordinary skill in the art will understand that the number and content of the entries can be different from those disclosed herein. In another embodiment, despite any depiction of the databases as tables, other formats including relational databases, object-based models, and/or distributed databases are used to store and manipulate the data types disclosed herein. Object methods or behaviors of a database can be used to implement various processes such as those disclosed herein. In another embodiment, the databases are, in a known manner, stored locally or remotely from a device that accesses data in such a database. In embodiments where there are multiple databases in the system 1700, the databases are integrated to communicate with each other for enabling simultaneous updates of data linked across the databases, when there are any updates to the data in one of the databases.

The method and the system 1700 disclosed herein can be configured to work in a network environment comprising one or more computers that are in communication with one or more devices via the network 1701. In an embodiment, the computers communicate with the devices directly or indirectly, via a wired medium or a wireless medium such as the Internet, a local area network (LAN), a wide area network (WAN) or the Ethernet, a token ring, or via any appropriate communications mediums or combination of communications mediums. Each of the devices comprises processors, examples of which are disclosed above, that are adapted to communicate with the computers. In an embodiment, each of the computers is equipped with a network communication device, for example, a network interface card, a modem, or other network connection device suitable for connecting to the network 1701. Each of the computers and the devices executes an operating system, examples of which are disclosed above. While the operating system may differ depending on the type of computer, the operating system provides the appropriate communications protocols to establish communication links with the network 1701. Any number and type of machines may be in communication with the computers.

The method and the system 1700 disclosed herein are not limited to a particular computer system platform, processor, operating system, or network. In an embodiment, one or more aspects of the method and the system 1700 disclosed herein are distributed among one or more computer systems, for example, servers configured to provide one or more services to one or more client computers, or to perform a complete task in a distributed system. For example, one or more aspects of the method and the system 1700 disclosed herein are performed on a client-server system that comprises components distributed among one or more server systems that perform multiple functions according to various embodiments. These components comprise, for example, executable, intermediate, or interpreted code, which communicate over the network 1701 using a communication protocol. The method and the system 1700 disclosed herein are not limited to be executable on any particular system or group of systems, and are not limited to any particular distributed architecture, network, or communication protocol.

The foregoing examples have been provided merely for the purpose of explanation and are in no way to be construed as limiting of the method and the system 1700 disclosed herein. While the method and the system 1700 have been described with reference to various embodiments, it is understood that the words, which have been used herein, are words of description and illustration, rather than words of limitation. Furthermore, although the method and the system 1700 have been described herein with reference to particular means, materials, and embodiments, the method and the system 1700 are not intended to be limited to the particulars disclosed herein; rather, the method and the system 1700 extend to all functionally equivalent structures, methods and uses, such as are within the scope of the appended claims. Those skilled in the art, having the benefit of the teachings of this specification, may effect numerous modifications thereto and changes may be made without departing from the scope and spirit of the method and the system 1700 disclosed herein in their aspects.

I claim:

1. A method for monitoring physical progress of a target object undergoing stem cell treatment for determining effectiveness of the stem cell treatment and adapting the stem cell treatment, the method comprising:
recording micro-movement data of one or more body parts of the target object, micro-movements of which need to be monitored on a time basis, the micro-movements monitored by three-dimensional motion sensors positioned within a wearable motion monitoring device configured as a body suit, wherein the three-dimensional motion sensors are positioned over the one or more body parts to be monitored, and wherein the micro-movement data comprises data on the micro-movements of the one or more body parts, and wherein the micro-movements of the one or more body parts comprise fine motor skills, gross motor skills, a fluid flow in a brain of the target object, an ultra-micro measurement for nerve activity of a spine of the target object, and a range of motion restored;
tabulating and summarizing the recorded micro-movement data collected by the three-dimensional motion sensors on the time basis comprising by day, week, and month; and
adapting the stem cell treatment using the tabulated and summarized micro-movement data by adapting a dosage and locations of administration of stem cells in the target object for improved care.

2. The method of claim 1, wherein the fine motor skills comprise hand-eye coordination, a range of motion of fingers of the target object, picking up objects between a thumb and a finger of the target object, and writing, and wherein the gross motor skills comprise movement of arms, hands, wrists, legs, feet, and toes of the target object.

3. The method of claim 1, wherein the micro-movement data is recorded corresponding to a predefined condition based stem cell treatment checklist.

4. A method for monitoring physical progress of a target object undergoing stem cell treatment for determining effectiveness of the stem cell treatment and adapting the stem cell treatment, the method employing a stem cell treatment monitoring system executable by at least one processor configured to execute computer program instructions for performing the method comprising:
rendering a predefined condition based stem cell treatment checklist on a graphical user interface provided by the stem cell treatment monitoring system deployed on an electronic device;
dynamically receiving, by the stem cell treatment monitoring system, neural activity data and motor activity data of the target object recorded by a plurality of sensors in operable communication with the stem cell treatment monitoring system on the electronic device, the sensors comprising:
neural sensors positioned proximal to a neural system of the target object, the neural sensors configured to measure the neural activity data comprising a change of a fluid flow and an ultra-micro measurement of nerve activity in the neural system of the target object; and
a wearable motion monitoring device configured as a body suit to conform to a body of the target object, the wearable motion monitoring device comprising motion sensors for measuring the motor activity data comprising data on micro-movements of one or more body parts of the body of the target object;
recording physiological condition parameters of the target object in the rendered predefined condition based stem cell treatment checklist by the stem cell treatment monitoring system using user inputs and the dynamically received neural activity data and the dynamically received motor activity data;
performing a baseline analysis of the physical progress of the target object undergoing the stem cell treatment by the stem cell treatment monitoring system based on the recorded physiological condition parameters in real time;
generating a physical progress report based on the baseline analysis of the physical progress of the target object by the stem cell treatment monitoring system for determining the effectiveness of the stem cell treatment, wherein the generated physical progress report is rendered on the graphical user interface displayed on the electronic device; and
generating and rendering a stem cell treatment plan based on the generated physical progress report of the target object on the graphical user interface displayed on the electronic device, by the stem cell treatment monitoring system.

5. The method of claim 4, further comprising receiving one or more color coded tomographic images and supplementary data of the target object from one or more external sources by the stem cell treatment monitoring system.

6. The method of claim 5, further comprising correlating the received one or more color coded tomographic images and the received supplementary data with the recorded physiological condition parameters to perform the baseline analysis of the physical progress of the target object undergoing the stem cell treatment.

7. The method of claim 5, wherein the supplementary data of the target object comprises medical reports, medical records, and physical therapy records of the target object.

8. The method of claim 4, wherein the neural sensors comprise a laser Doppler flow meter and electrodes configured to measure the neural activity data comprising information of a blood cell perfusion in microvasculature and the ultra-micro measurement of the nerve activity in a spine and a brain of the target object.

9. The method of claim 4, wherein the motion sensors of the wearable motion monitoring device comprise one of microelectromechanical system sensors, accelerometers, inertial sensors, gyroscopes, and any combination thereof.

10. The method of claim 4, wherein the generated stem cell treatment plan comprises type of stem cells to be administered to the target object, dosage of the stem cells to be administered to the target object, duration of the baseline analysis, area of administration of the stem cells, and physical therapy and behavioral therapy to be administered to the target object.

11. The method of claim 4, wherein the predefined condition based stem cell treatment checklist outlines criteria related to one of brain development, Lyme disease, a brain injury, and a spinal injury.

12. The method of claim 4, wherein the micro-movements of the one or more body parts of the body of the target object in the motor activity data measured by the motion sensors of the wearable motion monitoring device comprise fine motor skills, gross motor skills, and a range of motion restored.

13. The method of claim 12, wherein the fine motor skills comprise hand-eye coordination, a range of motion of fingers of the target object, picking up objects between a thumb and a finger of the target object, and writing, and wherein the gross motor skills comprise movement of arms, hands, wrists, legs, feet, and toes of the target object.

14. A system for monitoring physical progress of a target object undergoing stem cell treatment for determining effectiveness of the stem cell treatment and adapting the stem cell treatment, the system comprising:
a plurality of sensors comprising:
neural sensors positioned proximal to a neural system of the target object, the neural sensors configured to measure neural activity data comprising a change of a fluid flow and an ultra-micro measurement of nerve activity in the neural system of the target object; and
a wearable motion monitoring device configured as a body suit to conform to a body of the target object, the wearable motion monitoring device comprising motion sensors for measuring the motor activity data comprising data on micro-movements of one or more body parts of the body of the target object; and
an electronic device deploying a stem cell treatment monitoring system in operable communication with the sensors, the electronic device comprising:
a display screen for rendering a graphical user interface configured to display results of computations performed by modules of the stem cell treatment monitoring system;
a non-transitory computer readable storage medium configured to store computer program instructions defined by the modules of the stem cell treatment monitoring system; and
at least one processor communicatively coupled to the non-transitory computer readable storage medium, the at least one processor configured to execute the computer program instructions defined by the modules of the stem cell treatment monitoring system, the modules comprising:
a checklist rendering module configured to render a predefined condition based stem cell treatment checklist on the graphical user interface displayed on the display screen;
a data communication module configured to dynamically receive the neural activity data and the motor activity data of the target object recorded and transmitted by the sensors to the electronic device;
the data communication module further configured to record physiological condition parameters of the target object in the rendered predefined condition based stem cell treatment checklist using user inputs and the dynamically received neural activity data and the dynamically received motor activity data;
a baseline analysis module configured to perform a baseline analysis of the physical progress of the target object undergoing the stem cell treatment based on the recorded physiological condition parameters in real time;
a progress report generation module configured to generate a physical progress report based on the baseline analysis of the physical progress of the target object for determining the effectiveness of the stem cell treatment and to render the generated physical progress report on the graphical user interface displayed on the display screen; and
a treatment plan generation module configured to generate and render a stem cell treatment plan based on the generated physical progress report of the target object on the graphical user interface displayed on the display screen.

15. The system of claim 14, wherein the data communication module is further configured to receive one or more color coded tomographic images and supplementary data of the target object from one or more external sources.

16. The system of claim 15, wherein the baseline analysis module is further configured to perform the baseline analysis of the physical progress of the target object undergoing the stem cell treatment by correlating the received one or more color coded tomographic images and the received supplementary data with the recorded physiological condition parameters.

17. The system of claim 15, wherein the supplementary data of the target object comprises medical reports, medical records, and physical therapy records of the target object.

18. The system of claim 14, wherein the neural sensors comprise a laser Doppler flow meter and electrodes configured to measure the neural activity data comprising information of a blood cell perfusion in microvasculature and the ultra-micro measurement of the nerve activity in a spine and a brain of the target object.

19. The system of claim 14, wherein the motion sensors of the wearable motion monitoring device comprise one of microelectromechanical system sensors, accelerometers, inertial sensors, gyroscopes, and any combination thereof.

20. The system of claim 14, wherein the generated stem cell treatment plan comprises type of stem cells to be administered to the target object, dosage of the stem cells to be administered to the target object, duration of the baseline analysis, area of administration of the stem cells, and physical therapy and behavioral therapy to be administered to the target object.

21. The system of claim 14, wherein the predefined condition based stem cell treatment checklist outlines criteria related to one of brain development, Lyme disease, a brain injury, and a spinal injury.

22. The system of claim 14, wherein the micro-movements of the one or more body parts of the body of the target object in the motor activity data measured by the motion sensors of the wearable motion monitoring device comprise fine motor skills, gross motor skills, and a range of motion restored, and wherein the fine motor skills comprise hand-eye coordination, a range of motion of fingers of the target object, picking up objects between a thumb and a finger of the target object, and writing, and wherein the gross motor skills comprise movement of arms, hands, wrists, legs, feet, and toes of the target object.

23. A non-transitory computer readable storage medium having embodied thereon, computer program codes comprising instructions executable by at least one processor for monitoring physical progress of a target object undergoing stem cell treatment for determining effectiveness of the stem cell treatment and adapting the stem cell treatment, the computer program codes comprising:
a first computer program code for rendering a predefined condition based stem cell treatment checklist on a graphical user interface displayed on an electronic device;
a second computer program code for dynamically receiving neural activity data and motor activity data of the target object recorded by a plurality of sensors in operable communication with the electronic device, the sensors comprising:
neural sensors positioned proximal to a neural system of the target object, the neural sensors configured to measure the neural activity data comprising a change of a fluid flow and an ultra-micro measurement of nerve activity in the neural system of the target object; and
a wearable motion monitoring device configured as a body suit to conform to a body of the target object, the wearable motion monitoring device comprising motion sensors for measuring the motor activity data comprising data on micro-movements of one or more body parts of the body of the target object, wherein the micro-movements of the one or more body parts comprise fine motor skills, gross motor skills, and a range of motion restored;
a third computer program code for recording physiological condition parameters of the target object in the rendered predefined condition based stem cell treatment checklist using user inputs and the dynamically received neural activity data and the dynamically received motor activity data;
a fourth computer program code for performing a baseline analysis of the physical progress of the target object undergoing the stem cell treatment based on the recorded physiological condition parameters in real time;
a fifth computer program code for generating a physical progress report based on the baseline analysis of the physical progress of the target object for determining the effectiveness of the stem cell treatment, and rendering the generated physical progress report on the graphical user interface displayed on the electronic device; and
a sixth computer program code for generating and rendering a stem cell treatment plan based on the generated physical progress report of the target object on the graphical user interface displayed on the electronic device, wherein the generated stem cell treatment plan comprises type of stem cells to be administered to the target object, dosage of the stem cells to be administered to the target object, duration of the baseline analysis, area of administration of the stem cells, and physical therapy and behavioral therapy to be administered to the target object.

24. The non-transitory computer readable storage medium of claim 23, further comprising a seventh computer program code for receiving one or more color coded tomographic images and supplementary data of the target object from one or more external sources, wherein the supplementary data of the target object comprises medical reports, medical records, and physical therapy records of the target object.

25. The non-transitory computer readable storage medium of claim 23, wherein the fourth computer program code further comprises an eighth computer program code for correlating the received one or more color coded tomographic images and the received supplementary data with the recorded physiological condition parameters to perform the baseline analysis of the physical progress of the target object undergoing the stem cell treatment.
